(19) United States
(12) Patent Application Publication
(10) Pub. No.: US 2004/0146559 A1
(43) Pub. Date: Jul. 29, 2004

(54) Dosage forms having an inner core and outer shell with different shapes

(76) Inventors: Harry S. Sowden, Glenside, PA (US); Frank J. Bunick, Randolph, NJ (US); Gerard P. McNally, Berwyn, PA (US); Der-Yang Lee, Flemington, NJ (US); Martin Thomas, Lake Worth, FL (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/432,812

(22) PCT Filed: Sep. 28, 2002
(86) PCT No.: PCT/US02/31129

(51) Int. Cl. A61K 9/24

(52) U.S. Cl. 424/471

(57) ABSTRACT

A dosage form comprises an active ingredient, a core having an outer surface and a first shape and a shell having outer and inner surfaces and a second shape, in which the shell surrounds at least a portion of the core, and the first and second shapes are substantially different. In one embodiment the shell comprises at least about 80% of a flowable material selected from the group consisting of film formers, gelling polymers, thermoplastic materials, low melting hydrophobic materials, non-crystallizable sugars, non-crystallizable sugar-alcohols, and mixtures thereof. In another embodiment the shell is substantially free of pores having a diameter of 0.5 to 5.0 microns. In another embodiment, the core and shell each having a different number of planes of symmetry with respect to the same reference axis. In another embodiment, the distance from the core outer surface to the shell outer surface is different at two different points located on the core outer surface and the difference is greater than about 125 microns. Either the core, the shell, or a combination thereof may contain at least one active ingredient. The core and shell may each be molded or compressed. The core may also comprise an insert which may contain at least one active ingredient. The shape of the core may be chosen so as to permit modified release of active material in the core upon breach of the shell or provides a modified release profile for the active material in the core.

### BACKGROUND OF THE INVENTION

1. Field of the Invention

This invention relates to dosage forms such as pharmaceutical compositions having an inner core and an outer shell with different shapes. More particularly, this invention relates to dosage forms containing at least one active ingredient, in which the dosage form has an inner core and an outer shell in which the core shape and shell shape are substantially different. For example, the core and shell may each have a different number of axes of symmetry or different number of reflection lines with respect to the same reference axis. The outer surfaces of the core and shell may also have different topographies.

2. Background Information

A variety of dosage forms, such as tablets, capsules and gelcaps are known in the pharmaceutical arts. Tablets generally refer to relatively compressed powders in various shapes. Capsules are typically manufactured using a two piece gelatin shell formed by dipping a steel rod into an aqueous gelatin dispersion so that the gelatin coats the end of the rod. The gelatin is hardened by drying into two half-shells and the rod extracted. The hardened half-shells are then filled with a powder and the two halves joined together to form the capsule. (See generally, HOWARD C. ANSEL ET AL., Pharmaceutical Dosage Forms and Drug Delivery Systems (7th Ed. 1999).)

Film coated tablets are an improvement over uncoated tablets in terms of aesthetics, stability, and swallowability. One type of elongated, capsule-shaped film-coated tablet is commonly referred to as a “caplet.” Typical film coatings have a thickness from about 5 to about 50 microns, and comprise various film forming polymers such as cellulose ethers and the like. Typically, such polymers are applied to the tablets either from solution in organic solvents, or from aqueous dispersion via conventional spraying methods such as those disclosed in U.S. Pat. Nos. 4,973,480, and 6,113,945. Conventional spray-coating processes produce a relatively thin coating on the tablet surface so that the coated tablet has substantially the same overall shape as that of the uncoated tablet (core). Additionally, it is not commercially feasible to spray-coat a tablet with a different color coating on each end or face.

Sugar coated tablets, such as those disclosed in U.S. Pat. Nos. 2,925,365; 3,420,931; 4,456,629; and 3,361,631, and particularly those which have been polished, for example, with a top coat of carnuba wax, may typically possess higher surface gloss and thicker coatings than film coated tablets, however the sugar coating process is highly time consuming and costly, and the coatings thus prepared can disadvantageously retard the dissolution of the dosage forms. While sugar coatings are typically thicker than film coatings, and can have the effect of rounding the tablet edges, the overall shape of a sugar-coated tablet depends upon and is substantially the same as that of the uncoated core.

Gelatin-coated tablets, commonly known as geltabs and gelcaps, are an improvement on gelatin capsules and typically comprise a tablet coated with a glossy gelatinous shell. Several well known examples of gelcaps are McNeil-PPC, Inc.'s acetaminophen based products sold under the trade name Tylenol®. One category of methods for producing such geltabs and gelcaps involve dipping tablets, one half at a time, into coating solutions, which can be of two different colors, see e.g. U.S. Pat. Nos. 4,820,524, 5,538,125; 5,228,916; 5,436,026; 5,679,406; or dipping tablets of a first color halfway into a coating solution of a second color, see, e.g. U.S. Pat. No. 6,113,945. U.S. Pat. Nos. 5,942,034 and 6,195,911 describe additional methods and apparatuses for dip coating tablets. Another category of such methods involves shrink-fitting the capsule halves onto a tablet form. See, for example, U.S. Pat. Nos. 5,415,868; 6,126,767; 6,080,426; 5,460,824; 5,464,631; 5,795,588; 5,511,361; 5,609,010; 6,245,350; and WO 97/37629. Another method of producing gelcaps is via an enrobing process wherein two separate films made of gelatinous material are applied to opposite sides of a tablet by a pair of rotary dies, as disclosed for example, in U.S. Pat. Nos. 5,146,730 and 5,459,983.

Conventional methods for forming gelcaps are generally performed in a batchwise manner using a number of stand alone machines operating independently. Such batch processes typically include the unit operations of granulating, drying, blending, compacting (e.g., in a tablet press), film coating (e.g. by spraying in a coating pan), gelatin dipping, encapsulating or enrobing, drying, and printing. Gelcaps and geltabs prepared by either dipping or enrobing retain the essential shape characteristics of their uncoated cores.

Dipped gelcaps and geltabs may suffer from the limitations of variation in coating or shell thickness, and non-uniformity in color of the coating or shell.

All of the prior art methods for forming a shell on a core share the common limitation of having the shape of the shell depend upon and generally conform to the shape of the core. Other limitations shared by conventional encapsulation and enrobing processes include high cost and complexity, limitations on the thickness of the coating or shell, and the creation of raised seams between capsule halves and/or coatings. It would therefore be desirable to have dosage forms which have enhanced versatility for a number of applications, including dosage forms to deliver pharmaceuticals, nutritionals and/or confections, which may offer benefits of improved swallowability for an irregularly shaped substrate, or unique and pleasant aesthetic qualities that are valuable in the marketplace.

It is known to produce coatings on tablets by compression, to produce either multiple stacked layers, or core and shell configurations. Such coatings may have shapes which are substantially independent of the shape of the core, as disclosed for example in WO 00/18447. Commercially available compression coating machines are described in WO 89/11968. Modified release dosage forms prepared via compression are exemplified in U.S. Pat. Nos. 5,738,874 and 6,294,200, and WO 99/51209. It is possible via compression-coating to produce a 2-portion shell, which may function as a barrier, or release delaying coating, however compression-coated systems are limited by the shell thickness and shell composition as well as processing costs. Gunsel et al., “Compression-coated and layer tablets” in Pharmaceutical Dosage Forms—Tablets, edited by H. A. Lieberman, L. Lachman, J. B. Schwartz (2nd ed., rev. and expanded Marcel Dekker, Inc.), pp. 247-284, for example, discloses the thickness of compression coated shells is typically between 800 and 1200 microns and additionally states that “the advent of film coating dissipated much of the advantage of dry coating since larger quantities of tablets can be coated in a short time with film-formers dissolved in organic or aqueous solvents.” Typically, compressed coatings must contain a substantial amount of a compressible material. The compressed shell of WO 00/18447, for example, employs microcrystalline cellulose at a level of about 30%.

It is one object of this invention to provide a dosage form having an inner core and an outer shell, in which the inner core and outer shell have shapes which are substantially different. It is one feature of this invention that, in one embodiment, the core and shell have different numbers of planes of symmetry or reflection lines with respect to the same reference axis. It is another feature of this invention that, in another embodiment, the core has an outer surface and the shell has outer and inner surfaces such that the difference in the distances from the core outer surface to the outer surface of the shell measured at two different points on the core outer surface is greater than about 125 microns, preferably in the range of about 125-30,000 microns.

It is another object of this invention to provide a dosage form or pharmaceutical composition having a core with an outer surface with a first topography and a shell having an outer surface with a second topography, and the first topography is different from the second topography.

It is another object of this invention to provide a dosage form or pharmaceutical composition comprising a core having an outer surface and a shell having an inner surface and an outer surface, wherein the shell resides substantially conformally upon the core outer surface, such that the peaks and valleys of the inner surface of the shell substantially inversely correspond to the major peaks and valleys of the outer surface of the core, and the outer surface of the shell does not substantially conform to the major peaks and valleys of the outer surface of the core.

Other objects, features and advantages of this invention will be apparent to those skilled in the art from the detailed description of the invention provided herein.

### SUMMARY OF THE INVENTION

In one embodiment of this invention, the dosage form comprises: at least one active ingredient, a core having an outer surface and a first shape; and a shell having outer and inner surfaces and a second shape which is substantially different than the first shape, wherein the shell comprises at least about 80% of a flowable material selected from the group consisting of film formers, gelling polymers, thermoplastic materials, low melting hydrophobic materials, non-crystallizable sugars, non-crystallizable sugar alcohols, and mixtures thereof, and the shell surrounds at least a portion of the core.

In another embodiment of this invention, the dosage form comprises: at least one active ingredient; a core having an outer surface and a first shape; and a shell having outer and inner surfaces and a second shape which is substantially different than the first shape, wherein the shell is substantially free of pores having a pore diameter of 0.5 to 5.0 microns, and the shell surrounds at least a portion of the core.

In another embodiment of this invention, the dosage form comprises: at least one active ingredient; a core having an outer surface with a first topography; and a shell having an inner surface and an outer surface with a second topography which is different than the first topography, wherein at least one of the first or second topographies includes indentations or protrusions greater than about 20 microns in width, depth or height, and the shell surrounds at least a portion of the core.

In another embodiment of this invention, the dosage form comprises: at least one active ingredient; a core having an outer surface having indentations or protrusions greater than about 20 microns in width, depth, or height; and a shell having an inner surface and an outer surface, wherein the shell resides substantially conformally upon at least a portion of the core outer surface, such that the inner surface of the shell has protrusions and indentations corresponding substantially inversely to the major protrusions and indentations of the outer surface of the core, and the outer surface of the shell does not substantially conform to the major protrusions and indentations of the outer surface of the core.

In another embodiment of this invention, the dosage form comprises: at least one active ingredient; a core having an outer surface having indentations or protrusions; and a shell which surrounds at least a portion of the core, wherein the shell has an inner surface, an outer surface and a thickness, the ratio of the width of one or more indentations or protrusions in the core surface to the thickness of the shell at one or more locations is at least about 1:1, the shell resides substantially conformally upon the core outer surface such that the inner surface of the shell has protrusions and indentations correspond substantially inversely to the major indentations and protrusions of the outer surface of the core, and the outer surface of the shell does not substantially conform to the major protrusions and indentations of the outer surface of the core.

In another embodiment of this invention, the dosage form comprises: at least one active ingredient; a first core having an outer surface with a first topography; a second core having an outer surface with a second topography; and a shell having an inner surface and outer surface with a third topography which is different than the first topography, wherein at least one of the first, second, or third topographies includes indentations or protrusions greater than about 20 microns in width, depth or height, and the shell surrounds at least a portion of the core.

In another embodiment of this invention, the dosage form comprises: at least one active ingredient; a core comprising a first and second core portion having outer surfaces with a first and second topographies, respectively; a first shell portion having an outer surface with a third topography; and a second shell portion having an outer surface with a fourth topography, wherein at least one of the third or fourth shell surface topographies is different from the underlying core portion topography, at lease one of the first, second, third, or fourth topographies includes indentations or protrusions greater than about 20 microns in width, depth or height, and the shell surrounds at least a portion of the core.

In another embodiment of the invention, the core and shell each have a different number of planes of symmetry with respect to the same reference axis.

In another embodiment of the invention, the distance from the core outer surface to the shell outer surface is different at two different points located on the core outer surface, and the difference in distance is greater than about 125 microns.

In another embodiment of the invention, the difference in distance is in the range of about 125-30,000 microns.

In another embodiment of the invention, the shell comprises less than 10% by weight of a direct-compression filler-binder.

In another embodiment of the invention, the outer surface of the core displays written information, and the shell outer surface is transparent, semi-transparent or translucent.

In another embodiment of the invention, the outer surface of the shell displays written information.

In another embodiment of the invention, the shell is transparent, semi-transparent or translucent.

In another embodiment of the invention, the core and shell have different colors.

In another embodiment of the invention, the core is visually observable.

In another embodiment of the invention, the core, the shell, or both the core and shell comprise an active ingredient.

In another embodiment, only the core contains an active ingredient.

In another embodiment of the invention, the active ingredient is capable of dissolution, and dissolution of the active ingredient meets USP specifications for immediate release tablets containing the active ingredient.

In another embodiment of the invention, the core comprises a compressed dosage form.

In another embodiment of the invention, the core comprises a microelectronic device.

In another embodiment of the invention, the core comprises an insert.

In another embodiment of the invention, the insert is larger than the core in at least one dimension.

In another embodiment of the invention, at least a portion of the insert protrudes from the core.

In another embodiment of the invention, the insert comprises an active ingredient.

In another embodiment of the invention, the active ingredient is capable of dissolution, and dissolution of the active ingredient contained in the insert meets USP specifications for immediate release tablets containing the active ingredient.

In another embodiment of the invention, the insert comprises a microelectronic device.

In another embodiment of the invention, the outer surface of the shell is textured.

In another embodiment of the invention, the outer surface of the shell contains a prearranged pattern.

In another embodiment of the invention, the shell comprises one or more openings therein.

In another embodiment of the invention, the outer surface of the shell is substantially smooth.

In another embodiment of the invention, the shape of the core permits controlled release of one or more active ingredients contained in the core upon breach of the shell.

In another embodiment of the invention, the outer surface of the shell has a shape selected from the group consisting of spheres, ovoids, ellipses, and flattened derivatives thereof.

In another embodiment of the invention, the dosage form comprises a single core.

In another embodiment of the invention, the core and shell each have a major plane of symmetry, and the major plane of symmetry of the core is orthogonal to the major plane of symmetry of the shell.

In another embodiment of the invention, the core has an aperture therein defining an interior surface.

In another embodiment of the invention, the core is in the shape of a torus.

In another embodiment of the invention, the shell comprises first and second shell portions having first and second topographies respectively, and the first and second topographies are different.

In another embodiment of the invention, each of the first and second shell portions have an outer surface, and at least one of the outer surfaces comprises Braille symbols.

In another embodiment of the invention, the outer surface of the core contains indentations, intagliations, letters, symbols or a pattern.

In another embodiment of the invention, the shell covers a portion of the core, but does not substantially cover the indentations, intagliations, letters, symbols or pattern.

In another embodiment of the invention, a first shell portion covers the indentations, intagliations, letters, symbols or pattern but does not substantially cover the remaining portion of the core.

In another embodiment of the invention, a second shell portion covers the portion of the core which is not covered by the first shell portion.

In another embodiment of the invention, the outer surface of the core contains raised protrusions in the form of letters, symbols or a pattern.

In another embodiment of the invention, the shell covers a portion of the core, but does not substantially cover the raised protrusions.

In another embodiment of the invention, a first shell portion covers the raised protrusions, but does not substantially cover the remaining portion of the core.

In another embodiment of the invention, a second shell portion covers the portion of the core which is not covered by the first shell portion.

In another embodiment of the invention, the core outer surface is debossed or embossed with visual information and the shell outer surface is transparent, semi-transparent or translucent.

In another embodiment of the invention, the shell contains, based upon the total dry solids weight of the shell composition, from about 25 percent to about 80 percent of a film former; from about 0.10 percent to about 33 percent of a thickening agent; and from about 11 percent to about 60 percent of a plasticizer.

In another embodiment of the invention, the dosage form further comprises a second core.

In another embodiment of the invention, the shell outer surface has a topography which includes indentations or protrusions greater than about 20 microns in width, depth, or height.

In another embodiment of the invention, the outer surface of the shell contains a prearranged pattern.

In another embodiment of the invention, the prearranged pattern comprises Braille symbols.

In another embodiment of the invention, at least a portion of the shell comprises one or more openings therein.

In another embodiment of the invention, the shell comprises a plurality of openings therein.

In another embodiment of the invention, the openings form a prearranged pattern.

In another embodiment of the invention, the shell comprises first and second portions having first and second topographies, respectively, and the first and second topographies are different.

In another embodiment of the invention, at least a portion of the shell is transparent, semi-transparent, or translucent.

In another embodiment of the invention, the shell contains, based upon the total dry solids weight of the shell composition, from about 25 percent to about 80 percent of a film former; from about 0.10 percent to about 33 percent of a thickening agent; and from about 11 percent to about 60 percent of a plasticizer.

In another embodiment of the invention, the shell is substantially free of pores having a pore diameter of 0.5-5.0 microns.

### BRIEF DESCRIPTION OF THE DRAWINGS

FIGS. 1A and 1B are examples of dosage forms of this invention.

FIG. 1C is a side view of the shell and core portions of FIGS. 1A and 1B.

FIG. 1D is a side view of the shell and core portions in another embodiment of the invention.

FIG. 2 is a further depiction of the dosage form of this invention depicted in FIG. 1A.

FIG. 3 is another example of a dosage form of this invention.

FIGS. 4A and 4B depict top and cross-sectional views of another embodiment of the dosage form of this invention.

FIGS. 5A-5C depict top, cross-sectional and bottom views of another embodiment of the dosage form of this invention.

FIGS. 6A-6C depict top, cross-sectional and bottom views of another embodiment of the dosage form of this invention.

FIG. 7 is another example of a dosage form of this invention.

FIG. 8 is another example of a dosage form of this invention which comprises an insert.

FIG. 9 depicts a cross-sectional view of another embodiment of the dosage form of this invention.

FIG. 10 depicts a cross-sectional view of another embodiment of the dosage form of this invention.

FIG. 11 depicts a side view of another embodiment of the dosage form of this invention.

FIG. 12 depicts a side view of another embodiment of this invention.

### DETAILED DESCRIPTION OF THE INVENTION

As used herein, the term “dosage form” applies to any solid object, semi-solid, or liquid composition, designed to contain a specific pre-determined amount (i.e. dose) of a certain ingredient, for example an active ingredient as defined below. Suitable dosage forms may be pharmaceutical drug delivery systems, including those for oral administration, buccal administration, rectal administration, topical, transdermal, or mucosal delivery, or subcutaneous implants, or other implanted drug delivery systems; or compositions for delivering minerals, vitamins and other nutraceuticals, oral care agents, flavorants, and the like. Preferably the dosage forms of the present invention are considered to be solid, however they may contain liquid or semi-solid components. In a particularly preferred embodiment, the dosage form is an orally administered system for delivering a pharmaceutical active ingredient to the gastrointestinal tract of a human. In another preferred embodiment, the dosage form is an orally administered “placebo” system containing pharmaceutically inactive ingredients, and the dosage form is designed to have the same appearance as a particular pharmaceutically active dosage form, such as may be used for control purposes in clinical studies to test, for example, the safety and efficacy of a particular pharmaceutically active ingredient.

Suitable active ingredients for use in this invention include for example pharmaceuticals, minerals, vitamins and other nutraceuticals, oral care agents, flavorants and mixtures thereof. Suitable pharmaceuticals include analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, diuretics, expectorants, gastrointestinal agents, migraine preparations, motion sickness products, mucolytics, muscle relaxants, osteoporosis preparations, polydimethylsiloxanes, respiratory agents, sleep-aids, urinary tract agents and mixtures thereof.

Suitable oral care agents include breath fresheners, tooth whiteners, antimicrobial agents, tooth mineralizers, tooth decay inhibitors, topical anesthetics, mucoprotectants, and the like.

Suitable flavorants include menthol, peppermint, mint flavors, fruit flavors, chocolate, vanilla, bubblegum flavors, coffee flavors, liqueur flavors and combinations and the like.

Examples of suitable gastrointestinal agents include antacids such as calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotadine, ranitidine, cimetadine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate and loperamide; glycopyrrolate; antiemetics, such as ondansetron, analgesics, such as mesalamine.

In one embodiment of the invention, the active agent may be selected from bisacodyl, famotadine, ranitidine, cimetidine, prucalopride, diphenoxylate, loperamide, lactase, mesalamine, bismuth, antacids, and pharmaceutically acceptable salts, esters, isomers, and mixtures thereof.

In another embodiment, the active agent is selected from analgesics, anti-inflammatories, and antipyretics: e.g. non-steroidal anti-inflammatory drugs (NSAIDs), including propionic acid derivatives: e.g. ibuprofen, naproxen, ketoprofen and the like; acetic acid derivatives: e.g. indomethacin, diclofenac, sulindac, tolmetin, and the like; fenamic acid derivatives: e.g. mefanamic acid, meclofenamic acid, flufenamic acid, and the like; biphenylcarbodylic acid derivatives: e.g. diflunisal, flufenisal, and the like; and oxicams: e.g. piroxicam, sudoxicam, isoxicam, meloxicam, and the like. In a particularly preferred embodiment, the active agent is selected from propionic acid derivative NSAID: e.g. ibuprofen, naproxen, flurbiprofen, fenbufen, fenoprofen, indoprofen, ketoprofen, fluprofen, pirprofen, carprofen, oxaprozin, pranoprofen, suprofen, and pharmaceutically acceptable salts, derivatives, and combinations thereof. In another embodiment of the invention, the active agent may be selected from acetaminophen, acetyl salicylic acid, ibuprofen, naproxen, ketoprofen, flurbiprofen, diclofenac, cyclobenzaprine, meloxicam, rofecoxib, celecoxib, and pharmaceutically acceptable salts, esters, isomers, and mixtures thereof.

In another embodiment of the invention, the active agent may be selected from pseudoephedrine, phenylpropanolamine, chlorpheniramine, dextromethorphan, diphenhydramine, astemizole, terfenadine, fexofenadine, loratadine, desloratadine, doxilamine, norastemizole, cetirizine, mixtures thereof and pharmaceutically acceptable salts, esters, isomers, and mixtures thereof.

Examples of suitable polydimethylsiloxanes, which include, but are not limited to dimethicone and simethicone, are those disclosed in U.S. Pat. Nos. 4,906,478, 5,275,822, and 6,103,260. As used herein, the term “simethicone” refers to the broader class of polydimethylsiloxanes, including but not limited to simethicone and dimethicone.

The active ingredient or ingredients are present in the dosage form in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dose regime, the age and weight of the patient, and other factors must be considered, as known in the art. In one embodiment, the dosage form comprises at least about 25 weight percent, e.g. at least about 50 weight percent of one or more active ingredients.

In certain embodiments in which modified release of the active ingredient is desired, at least a portion of the active ingredient may optionally be coated with a release-modifying coating, as known in the art. Examples of suitable release modifying coatings are described in U.S. Pat. Nos. 4,173,626; 4,863,742; 4,980,170; 4,984,240; 5,286,497, 5,912,013; 6,270,805; and 6,322,819. Preferably, the particle coating may comprise about 10-100 weight percent (based on the weight of the coating) of a film former; optionally up to about 50 weight percent based on the weight of the coating of a pore former; and optionally up to about 30 weight percent of various adjuvants or excipients such as plasticizers etc. The particles may be coated using conventional coating technology which is well known to those skilled in the art including microencapsulation techniques such as coaccervation, spray-drying, and fluidized bed coating including rotor coating and wurster coating Commercially available modified release active ingredients may also be employed. Accordingly, all or a portion of one or more active ingredients may be coated with a release-modifying material.

If the active ingredient has an objectionable taste, and the dosage form is intended to be chewed or disintegrated in the mouth prior to swallowing, the active ingredient may be coated with a taste masking coating, as known in the art. Examples of suitable taste masking coatings are described in U.S. Pat. No. 4,851,226, U.S. Pat. No. 5,075,114, and U.S. Pat. No. 5,489,436. Commercially available taste masked active ingredients may also be employed. For example, acetaminophen particles which are encapsulated with ethylcellulose or other polymers by a coaccervation process may be used in the present invention as described above

The core of the present invention may be prepared by any suitable method, including for example compression and molding, and depending on the method by which it is made, typically comprises, in addition to the active ingredient, a variety of excipients (inactive ingredients which may be useful for conferring desired physical properties to the core or dosage form).

In embodiments in which the core is prepared by compression, suitable excipients for compression include fillers, binders, disintegrants, lubricants, glidants, and the like.

Suitable fillers include water-soluble compressible carbohydrates such as sugars, which include dextrose, sucrose, isomaltalose, fructose, maltose, and lactose, polydextrose, sugar-alcohols, which include mannitol, sorbitol, isomalt, maltitol, xylitol, erythritol, starch hydrolysates, which include dextrins, and maltodextrins, and the like, water insoluble plasticly deforming materials such as microcrystalline cellulose or other cellulosic derivatives, water-insoluble brittle fracture materials such as dicalcium phosphate, tricalcium phosphate and the like and mixtures thereof.

Suitable binders include dry binders such as polyvinyl pyrrolidone, hydroxypropylmethylcellulose, and the like; wet binders such as water-soluble polymers, including hydrocolloids such as alginates, agar, guar gum, locust bean, carrageenan, tara, gum arabic, tragacanth, pectin, xanthan, gellan, maltodextrin, galactomannan, pusstulan, laminarin, scleroglucan, gum arabic, inulin, pectin, whelan, rhamsan, zooglan, methylan, chitin, cyclodextrin, chitosan, polyvinyl pyrrolidone, cellulosics, starches, and the like; and derivatives and mixtures thereof.

Suitable disintegrants include sodium starch glycolate, cross-linked polyvinylpyrrolidone, cross-linked carboxymethylcellulose, starches, microcrystalline cellulose, and the like.

Suitable lubricants include long chain fatty acids and their salts, such as magnesium stearate and stearic acid, talc, and waxes.

Suitable glidants include colloidal silicon dioxide, and the like.

The dosage form of the invention may also incorporate pharmaceutically acceptable adjuvants, including, for example, preservatives, high intensity sweeteners such as aspartame, acesulfame potassium, cyclamate, saccharin, sucralose, and the like; and other sweeteners such as dihydroalcones, glycyrrhizin, Monellin™, stevioside, Talin™, and the like; flavors, antioxidants, surfactants, and coloring agents.

In embodiments in which it is desired for the active ingredient to be absorbed into the systemic circulation of an animal, the active ingredient or ingredients are preferably capable of dissolution upon contact with a fluid such as water, gastric fluid, intestinal fluid or the like. In one embodiment, the dissolution characteristics of the active ingredient meet USP specifications for immediate release tablets containing the active ingredient. For example, for acetaminophen tablets, USP 24 specifies that in pH 5.8 phosphate buffer, using USP apparatus 2 (paddles) at 50 rpm, at least 80% of the acetaminophen contained in the dosage form is released therefrom within 30 minutes after dosing, and for ibuprofen tablets, USP 24 specifies that in pH 7.2 phosphate buffer, using USP apparatus 2 (paddles) at 50 rpm, at least 80% of the ibuprofen contained in the dosage form is released therefrom within 60 minutes after dosing. See USP 24, 2000 Version, 19-20 and 856 (1999). In another embodiment, the dissolution characteristics of the active ingredient are modified: e.g. controlled, sustained, extended, retarded, prolonged, delayed and the like.

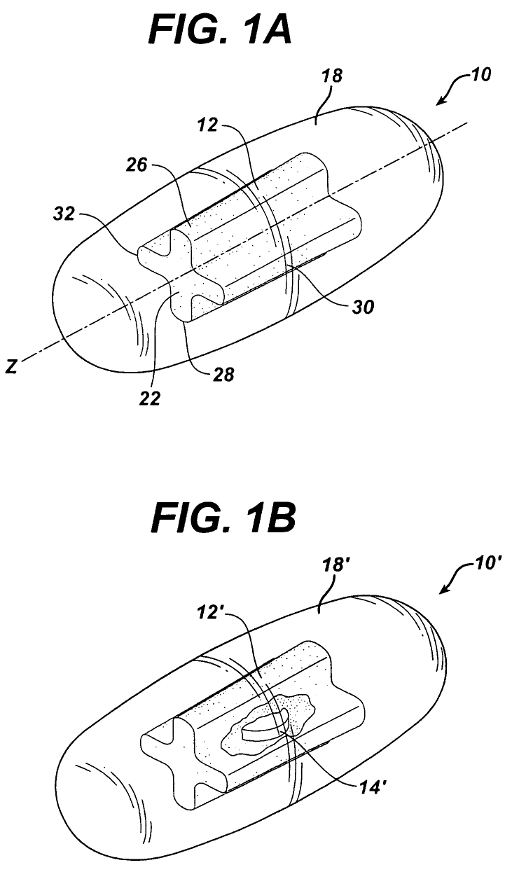

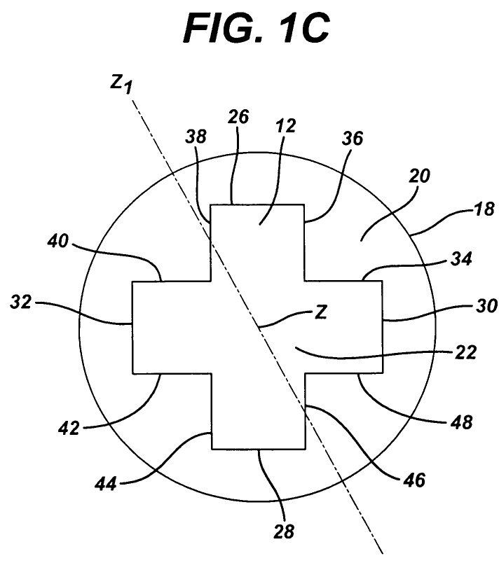

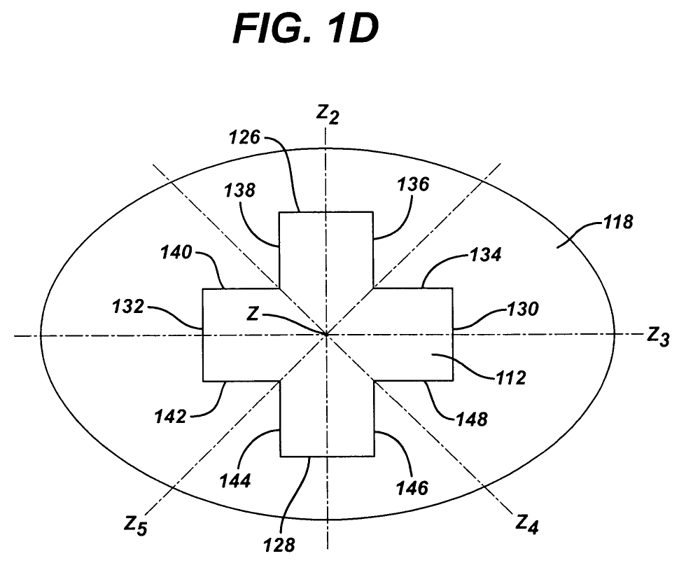

An overall understanding of the dosage form of this invention may be obtained by reference to FIGS. 1A and 1B. In FIG. 1A, a dosage form 10 is depicted which comprises a shell 18 (which may be a molded shell) having a shape which surrounds the outside surface of a core 12 (which may be a molded core or a compressed core or a hard or soft capsule, or any substantially solid edible form) having a different shape than the shell 18. FIG. 1B illustrates an alternative dosage form 10′ which comprises a shell 18′ (which may be a molded shell) having a shape which surrounds the outside surface of a core 12′ (which may be a molded core or a compressed core or a hard or soft capsule, or any substantially solid edible form) having a different shape than the shell 18′. Core 12′ contains an insert 14′ as is further described herein. It will be understood that the shapes of the core and shell in FIGS. 1A and 1B are merely illustrative, and are not meant to limit this invention in any way.

In a first embodiment of the invention, the shapes of core and the shell are substantially different in the dosage form of this invention. As used herein, the term “substantially different” refers to shapes which would be recognized by those skilled in the art, upon visual observation, as having a different number of sides or, if having the same number of sides, having different angles of intersection of such sides or having different degrees of curvature of such sides. Thus, the two dimensional contours of the shell outer surface and core outer surface are geometrically distinct from each other in at least one cross-section through any plane of the dosage form.

In one preferred embodiment of the invention, the substantially different shapes of the core and shell are readily apparent because the core and shall each have a different number of planes of symmetry with respect to the same reference axis. As will be recognized by those skilled in the art, the term “planes of symmetry” as used herein refers to planes which may be drawn through a given object such that the portions of the object on each side of the plane are mirror images of each other. This may also be referred to as the “reflection line” or “mirror line.” In this embodiment, a given reference line (say the X axis) is chosen. If the core has a different number of planes of symmetry with respect to the X axis than the number of planes of symmetry of the shell with respect to the X axis, then the shapes of the core and shell are considered to be substantially different.

For example, FIG. 1A depicts a dosage form according to the invention comprising a core 12 inside a shell 18. Shell 18 has the shape of an elliptical or ovular spheroid. Single continuous side or face 20 is the external face of this spheroid. In contrast, core 12 has end faces 22 and 24. FIG. 1C is an end view of the dosage form viewing end face 22 inside shell 18 (which in this embodiment has a circular shape from the end view). As shown in FIG. 1C, core 12 has additional top and bottom faces 26 and 28, side faces 30 and 32, and intermediate faces 34, 36, 38, 40, 42, 44, 46 and 48. Using the Z centerline as the reference axis, there are an infinite number of planes of symmetry or mirror lines of shell 18 about the Z axis. Plane Z₁ (shown in dashed lines) is one such mirror line for shell 18. However plane Z₁ is not a mirror line for core 12. Accordingly, the shapes of core 12 and shell 18 are substantially different.

In other embodiments of the invention, for example as shown in FIG. 1D, the shell need not have a circular cross-section and thus there are not an infinite number of planes of symmetry or mirror lines of the shell about the centerline. More particularly, FIG. 1D depicts a shell 118 in an end view (the shell in this embodiment has an oval shape from the end view) and the core 112 in an end view of end face 122. Core 112 has additional top and bottom faces 126 and 128, side faces 130 and 132, and intermediate faces 134, 136, 138, 140, 142, 144, 146 and 148. Using the Z centerline as the reference axis, there are two of planes of symmetry or mirror lines, Z₂ and Z₃, of shell 18 about the Z axis. However, there are four planes of symmetry or mirror lines, Z₂, Z₃, Z₄, and Z₅, of core 112 about the Z axis. Accordingly, the shapes of core 112 and shell 118 are substantially different.

In another embodiment of the invention, the shell, core or both have no planes of symmetry. In this embodiment the shapes of the shell and the core are substantially different.

In another preferred embodiment of the invention, the substantially different shapes of the core and shell are readily apparent because the distance measured from the outer surface of the core to the outer shell surface is different at two different points located on the outer surface of the core, and the difference in the distances is greater than about 125 microns, preferably in the range of about 125-30,000 microns, more preferably about 125-20,000 microns, most preferably about 150-10,000 microns. The “measured distance” referred to herein refers to a vector line emanating from a point on the core outer surface and contacting the shell outer surface at a point. For example, in FIG. 2 vector line R₁ extends a distance A from a point on the exterior surface of the core to a point on the exterior surface of the shell. Similarly, vector line R₂ extends a distance B from a different point on the exterior surface of the core to a different point on the exterior surface of the shell. The difference between the distances A and B is greater than 125 microns, preferably about 125-30,000 microns, more preferably about 125-20,000 microns, most preferably about 150-10,000 microns. Accordingly, the core and shell have substantially different shapes.

In another embodiment of the invention, the core has an outer surface with a first topography; and the shell has an inner surface, and an outer surface with a second topography which is different than the first topography, wherein at least one of the first or second topographies includes indentations or protrusions greater than about 20 microns in width, depth or height, and the shell surrounds at least a portion of the core.

In one embodiment of the invention, as depicted in FIGS. 1A and 1B, the shell may be transparent, semi-transparent or translucent, and the core may be visually observable through the shell. In such embodiments, the core may display written information on its outer surface which may be observable through the shell. In other embodiments, the outer surface of the shell may display written information.

In another embodiment of the invention, the outer surface of the core may contain an embossed (raised) or debossed (indented) design, such as for example lettering or a graphic or logo. For example, the outer surface of the core may contain indentations, intagliations, letters, symbols or a pattern.

In another embodiment of the invention, the shell may cover a portion of the core. For example, the core may comprise a raised design. The shell may cover only that portion of the core not containing the raised design, leaving the raised designed exposed for view.

In another embodiment of the invention, the shell covers a portion of the core, but does not substantially cover the indentations, intagliations, letters, symbols or pattern on the core.

In another embodiment of the invention, a first shell portion covers the indentations, intagliations, letters, symbols or pattern on the core, but does not substantially cover the remaining portion of the core.

In another embodiment of the invention, a second shell portion covers the portion of the core which is not covered by the first shell portion.

In another embodiment of the invention, the outer surface of the shell may display written information.

In another embodiment of the invention, the outer surface of the shell may be textured. In one such embodiment, the outer surface of the core may be substantially smooth.

In another embodiment of the invention, the outer surface of the shell may contain a prearranged pattern.

In another embodiment of the invention, the outer surface of the shell may be substantially smooth.

In another embodiment of the invention, the shell may have one or more openings therein.

In another embodiment of this invention, the shell does not entirely surround the core. For example, the core may have one or more protrusions which protrude through a portion of the shell.

The core (or substrate) may be any solid or semi-solid form. The core may prepared by any suitable method, for example the core be a compressed dosage form, or may be molded. As used herein, “substrate” refers to a surface or underlying support, upon which another substance resides or acts, and “core” refers to a material which is at least partially enveloped or surrounded by another material. For the purposes of the present invention, the terms may be used interchangeably: i.e. the term “core” may also be used to refer to a “substrate.” Preferably, the core comprises a solid, for example, the core may be a compressed or molded tablet, hard or soft capsule, suppository, or a confectionery form such as a lozenge, nougat, caramel, fondant, or fat based composition. In certain other embodiments, the core may be in the form of a semi-solid or a liquid in the finished dosage form.

In one embodiment, the core has one or more major faces. The core may be in a variety of different shapes. For example, in one embodiment the core may be in the shape of a truncated cone. In other embodiments the core may be shaped as a polyhedron, such as a cube, pyramid, prism, or the like; or may have the geometry of a space figure with some non-flat faces, such as a cone, cylinder, sphere, torus, or the like. Exemplary core shapes which may be employed include tablet shapes formed from compression tooling shapes described by “The Elizabeth Companies Tablet Design Training Manual” (Elizabeth Carbide Die Co., Inc., p.7 (McKeesport, Pa.) (incorporated herein by reference) as follows (the tablet shape corresponds inversely to the shape of the compression tooling):

1. Shallow Concave.

2. Standard Concave.

3. Deep Concave.

4. Extra Deep Concave.

5. Modified Ball Concave.

6. Standard Concave Bisect.

7. Standard Concave Double Bisect.

8. Standard Concave European Bisect.

9. Standard Concave Partial Bisect.

10. Double Radius.

11. Bevel & Concave.

12. Flat Plain.

13. Flat-Faced-Beveled Edge (F.F.B.E.).

14. F.F.B.E. Bisect.

15. F.F.B.E. Double Bisect.

16. Ring.

17. Dimple.

18. Ellipse.

19. Oval.

20. Capsule.

21. Rectangle.

22. Square.

23. Triangle.

24. Hexagon.

25. Pentagon.

26. Octagon.

27. Diamond.

28. Arrowhead.

29. Bullet.

30. Barrel.

31. Half Moon.

32. Shield.

33. Heart.

34. Almond.

35. House/Home Plate.

36. Parallelogram.

37. Trapezoid.

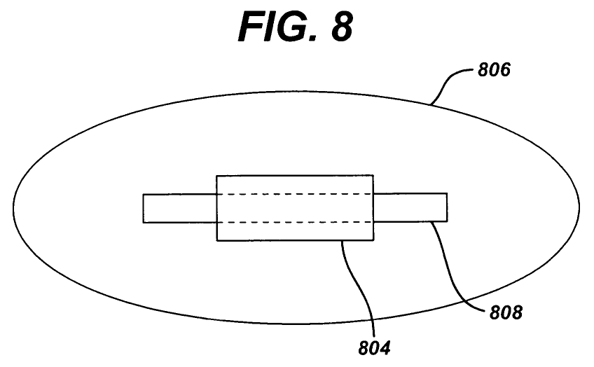

38. FIG. 8/Bar Bell.

39. Bow Tie.

40. Uneven Triangle.

The core surface may be substantially smooth, i.e. may have indentations or protrusions at the microscopic level on the order of less than about 20 microns in width, depth, or height. Alternately the core surface may be textured, i.e. having indentations or protrusions greater than about 20 microns, e.g. greater than about 50 microns, or greater than about 100 microns, say greater than about 1000 microns in width, depth, or height. The indentations or protrusions may be up to about 30,000 microns, e.g. up to about 2,000 microns in width, depth, or height. In embodiments wherein the core surface is textured, the outer surface of the core may contain an embossed (raised) or debossed (indented) design. For example, the outer surface of the core may contain indentations, intagliations, letters, symbols or a pattern such as a graphic or logo.

In one embodiment of this invention, the core is a compressed dosage form: i.e. a tablet, obtained from a compressed powder. The powder may preferably comprise an active ingredient, and optionally comprise various excipients, such as binders, disintegrants, lubricants, fillers and the like, as is conventional, or the powder may comprise other particulate material of a medicinal or non-medicinal nature, such as inactive placebo blends for tableting, confectionery blends, and the like. One particular formulation comprises active ingredient, powdered wax (such as shellac wax, microcrystalline wax, polyethylene glycol, and the like), and optionally disintegrants and lubricants and is described in more detail in pending U.S. patent application Ser. No. 09/966,493 at pages, 4-11, the disclosure of which is incorporated herein by reference.

The core may optionally comprise a sub-core (which may also be referred to as an “insert”), which may be made by any method, for example compression or molding, and which may optionally contain one or more active ingredients.

The core or sub-core may optionally be at least partially covered by a compressed, molded, or sprayed sub-coating. However, in one preferred embodiment, the core may be substantially free of the subcoating: i.e. there is no subcoating located between the outer surface of the core and the inner surface of the shell.

In one embodiment of the invention, the dosage forms of this invention comprise a core made from a blend of powders having an average particle size of about 50 to about 500 microns, e.g. about 100 to about 500 microns. In one embodiment, the active ingredient has an average particle size of about 50 to about 500 microns, e.g. about 100 to about 500 microns. In another embodiment, at least one excipient has an average particle size of about 50 to about 500 microns, e.g. about 100 to about 500 microns. In one such embodiment, a major excipient, i.e. an excipient comprising at least 50% by weight of the core, has an average particle size of about 50 to about 500 microns, e.g. about 100 to about 500 microns. Particles in this size range are particularly useful for direct compression processes. In a preferred embodiment of the invention, the core may be prepared by a direct compression process.

In one such embodiment of the invention, the core is a directly compressed tablet, made from a powder which is substantially free of water soluble polymeric binders and hydrated polymers. This composition is advantageous for maintaining an immediate release dissolution profile, minimizing processing and material costs, and providing for optimal physical and chemical stability of the dosage form.

In embodiments in which the core is prepared by direct compression, the materials comprising the core, e.g. the active ingredient or ingredients and excipients, are blended together, preferably as dry powders, and fed into an apparatus that applies pressure and forms a core. Any suitable compacting apparatus may be used, including for example a roller compactor such as a chilsonator or drop roller; or a conventional tablet press. Preferably, the core is formed by compaction using a rotary tablet press as known in the art. In a rotary tablet press, a metered volume of powder is filled into a die cavity, which rotates as part of a “die table” from the filling position to a compaction position where the powder is compacted between an upper and a lower punch to an ejection position where the resulting tablet is pushed from the die cavity by the lower punch. The direct compression process enables the minimization or elimination of water-soluble, non-saccharide polymeric binders such as polyvinyl pyrrolidone, alginates, hydroxypropyl cellulose, hydroxypropylmethylcellulose, hydroxyethylcellulose, and the like, which can have an adverse effect on dissolution.

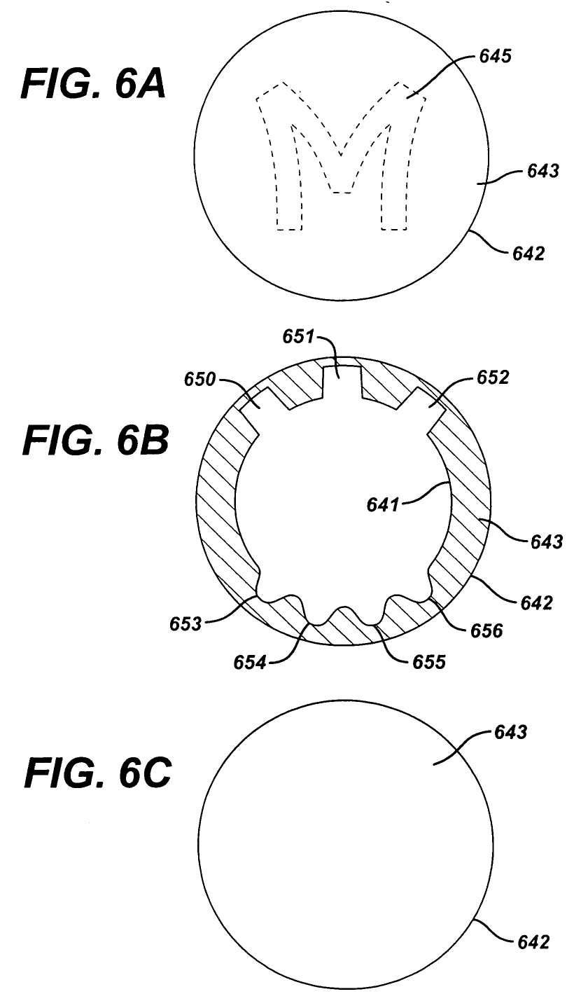

In a preferred embodiment, the core is prepared by the compression methods and apparatus described in copending U.S. application Ser. No. 09/966,509, pages 16-27, the disclosure of which is incorporated herein by reference. Specifically, the core is made using a rotary compression module comprising a fill zone, insertion zone, compression zone, ejection zone, and purge zone in a single apparatus having a double row die construction as shown in FIG. 6 of U.S. application Ser. No. 09/966,509. The dies of the compression module are preferably filled using the assistance of a vacuum, with filters located in or near each die. The purge zone of the compression module includes an optional powder recovery system to recover excess powder from the filters and return excess powder to the dies.

In another embodiment, the core is prepared by a wet-granulation method, in which the active ingredient or ingredients, appropriate excipients, and a solution or dispersion of a wet binder (e.g. an aqueous cooked starch paste, or solution of polyvinyl pyrrolidone) are mixed and granulated. Suitable apparatuses for wet granulation include low shear, e.g. planetary mixers; high shear mixers; and fluid beds, including rotary fluid beds. The resulting granulated material is dried, and optionally dry-blended with further ingredients, e.g. adjuvants and/or excipients such as for example lubricants, colorants, and the like. The final dry blend is then suitable for compression by the methods described in the previous paragraphs.

Methods for direct compression and wet granulation processes are known in the art, and are described in detail in, for example, Lachman, et al., The Theory and Practice of Industrial Pharmacy, Chapter 11 (3rd ed. 1986).

In another embodiment, the core is prepared by thermal setting molding using the method and apparatus described in copending U.S. patent application Ser. No. 09/966,450, pages 57-63, the disclosure of which is incorporated herein by reference. In this embodiment, the core is formed by injecting a starting material in flowable form into a molding chamber. The starting material preferably comprises an active ingredient and a thermal setting material at a temperature above the melting point of the thermal setting material but below the decomposition temperature of the active ingredient. The starting material is cooled and solidifies in the molding chamber into a shaped form (i.e., having the shape of the mold).

According to this method, the starting material must be in flowable form. For example, it may comprise solid particles suspended in a molten matrix, for example a polymer matrix. The starting material may be completely molten or in the form of a paste. The starting material may comprise an active ingredient dissolved in a molten material. Alternatively, the starting material may be made by dissolving a solid in a solvent, which solvent is then evaporated from the starting material after it has been molded.

In another embodiment, the core is prepared by thermal cycle molding using the method and apparatus described in copending U.S. patent application Ser. No. 09/966,497, pages 27-51, the disclosure of which is incorporated herein by reference. In this embodiment, the core is formed by injecting a starting material in flowable form into a heated molding chamber. The starting material preferably comprises an active ingredient and a thermoplastic material at a temperature above the set temperature of the thermoplastic material but below the decomposition temperature of the active ingredient. The starting material is cooled and solidifies in the molding chamber into a shaped form (i.e., having the shape of the mold).

The starting material may comprise any edible material which is desirable to incorporate into a shaped form, including active ingredients such as those active ingredients previously described with respect to the core, nutritionals, vitamins, minerals, flavors, sweeteners, and the like. Preferably, the starting material comprises an active ingredient and a thermal setting material. The thermal setting material may be any edible material that is flowable at a temperature between about 37 and about 250° C., and that is a solid or semi-solid at a temperature between about −10° C. and about 35° C. Preferred thermal setting materials include water-soluble polymers such as polyalkylene glycols, polyethylene oxides and derivatives, and sucrose esters; fats such as cocoa butter, hydrogenated vegetable oil such as palm kernel oil, cottonseed oil, sunflower oil, and soybean oil; free fatty acids and their salts; mono- di- and triglycerides, phospholipids, waxes such as carnuba wax, spermaceti wax, beeswax, candelilla wax, shellac wax, microcrystalline wax, and paraffin wax; fat-containing mixtures such as chocolate; sugar in the form on an amorphous glass such as that used to make hard candy forms, sugar in a supersaturated solution such as that used to make fondant forms; low-moisture polymer solutions such as mixtures of gelatin and other hydrocolloids at water contents up to about 30% such as those used to make “gummi” confection forms. In a particularly preferred embodiment, the thermal setting material is a blend of fats and mono- and diglycerides.

In another embodiment, the core may be a hollow or evacuated core. For example, the core may be an empty capsule shell. Alternatively, a hollow core may be prepared for example by molding. In one such method, flowable material is injected into a mold cavity, the cavity is brought to a temperature at which the outer surface of the core (which is in contact with the mold) begins to solidify or set. The excess flowable material from the center of the core is then withdrawn from the mold using suitable means, for example a piston pump. In another such method, an empty capsule is used as a sub-core, and a coating layer is formed thereon by methods known in the art such as, for example, spray-coating, dip-coating, or thermal cycle molding as described in copending U.S. patent application Ser. No. 09/966,497, pages 27-51, the disclosure of which is incorporated herein by reference.

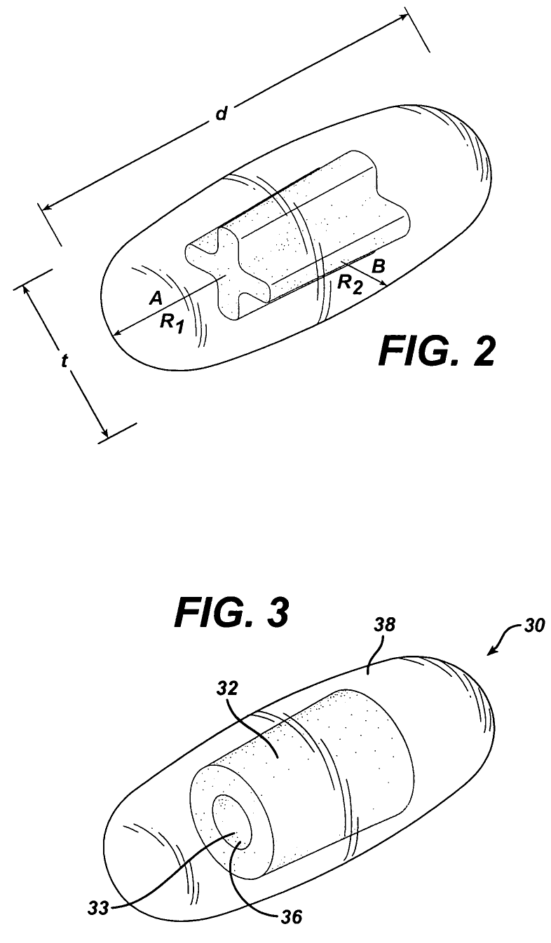

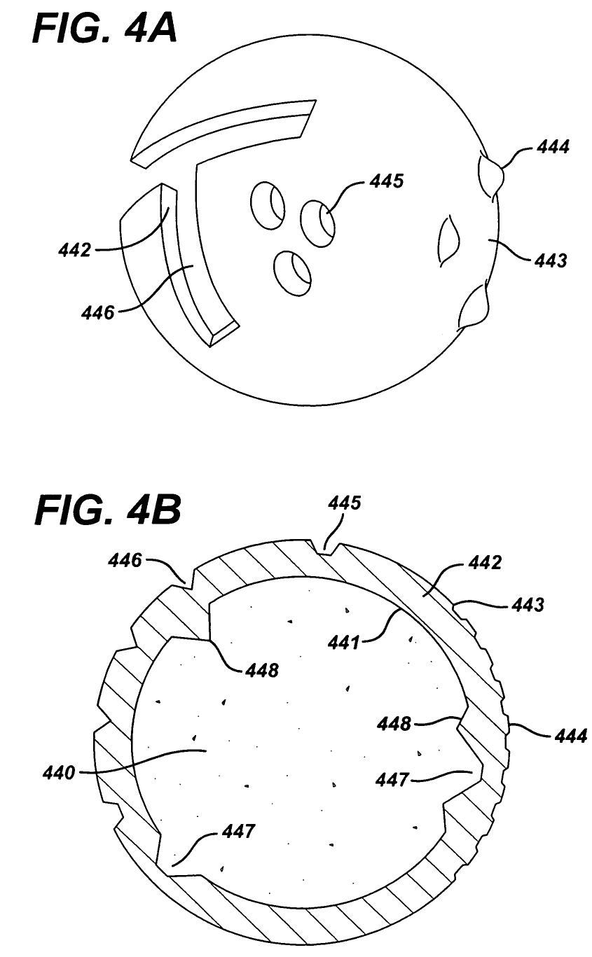

In the thermal cycle molding method and apparatus of U.S. patent application Ser. No. 09/966,497 a thermal cycle molding module having the general configuration shown in FIG. 3 therein is employed. The thermal cycle molding module 200 comprises a rotor 202 around which a plurality of mold units 204 are disposed. The thermal cycle molding module includes a reservoir 206 (see FIG. 4) for holding flowable material to make the core. In addition, the thermal cycle molding module is provided with a temperature control system for rapidly heating and cooling the mold units. FIGS. 55 and 56 depict the temperature control system 600.

In this embodiment, the mold units preferably comprise center mold assemblies 212 and upper mold assemblies 214 as shown in FIG. 26C, which mate to form mold cavities having the desired shape of the core. As rotor 202 rotates, the opposing center and upper mold assemblies close. Core flowable material, which is heated to a flowable state in reservoir 206, is injected into the resulting mold cavities. The temperature of the core flowable material is then decreased, hardening the core flowable material into cores. The mold assemblies open and eject the cores.

In certain embodiments of the invention, the core may further comprise a subcoating, applied by any method, for example spraying, compression, or molding. In certain other embodiments of the invention, the core may be substantially free of a subcoating.

In another embodiment of the invention, the core contains at least in part one or more inserts. The inserts can be made in any shape or size. For instance, irregularly shaped inserts can be made, that is shapes having no more than one axis of symmetry. Cylindrically shaped inserts may also be made. The insert may be prepared by conventional methods, such as panning or compression. In a preferred embodiment, the insert is prepared using the above described thermal setting method and apparatus described in copending U.S. patent application Ser. No. 09/966,450, pages 57-63.

In one embodiment of the invention, the insert may have an average diameter from about 100 to about 1000 microns. In another embodiment of this invention, the insert may have an average diameter or thickness from about 10% to about 90% of the diameter or thickness of the core. In yet another embodiment of this invention, the core may comprise a plurality of inserts.

In another embodiment, the insert may have an average diameter, length, or thickness greater than about 90% of the diameter or thickness of the core, for example the insert may have an average length greater than about 100% of the thickness of the core.

In another embodiment of the invention, the core, the insert (if employed) or both may comprise a microelectronic device (e.g. an electronic “chip”) which may be used as an active component or to control, for example, the rate of release of active ingredients within the core or insert in response to an input signal. Examples of such microelectronic devices are as follows:

(1) Integrated, self-regulating responsive therapeutic devices including biosensors, electronic feedback and drug/countermeasure release devices which are fully integrated. Such devices eliminate the need for telemetry and human intervention, and are disclosed, for example, at www.chiprx.com/products.html, which is incorporated herein by reference;

(2) Miniaturized diagnostic imaging systems which comprise a swallowable capsule containing a video camera, and are disclosed, for example, at www.givenimaging.com/usa/default.asp, which is incorporated herein by reference;

(3) Subcutaneous glucose monitors which comprise implantable or insertable sensor devices which detect changes in glucose concentration within intestinal fluid, and communicate to an external detector and data storage device. Such devices are disclosed, for example, at www.applied-medical.co.uk/glucose.htm, which is incorporated herein by reference;

(4) Microdisplay vision aid devices encapsulated in an artificial intraocular lens. Such devices include a receiver for power supply, data and clock recovery, and a miniature LED array flip-chip bonded to a silicon CMOS driver circuit and micro optics, and are disclosed, for example, at http://ios.oe.uni-duisberg.de/e/, which is incorporated herein by reference. The microdisplay device receives a bit-stream+energy wireless signal from a high dynamic range CMOS camera placed outside the eye which generates a digital black & white picture which is converted by a digital signal processing unit (DAP) into a serial bit-stream with a data rate of approximately 1 Mbit/s. The image is projected onto the retina;

(5) Microchips used to stimulate damaged retinal cells, allowing them to send visual signals to the brain for patients with macular degeneration or other retinal disorders. The chip is 2 mm×25 microns, and contains approximately 5,000 microscopic solar cells (“microphotodiodes”), each with its own stimulating electrode. These microphotodiodes convert the light energy from images into electrical chemical impulses that stimulate the remaining functional cells of the retina in patients with AMD and RP. Such microchips are disclosed, for example, at www.optobionics.com/artificialretina.htm, which is incorporated herein by reference;

(6) Disposable “smart needles” for breast biopsies which display results in real time. The device fits into a 20 to 21 gauge disposable needle that is connected to a computer, as the needle is inserted into the suspicious lesion. The device measures oxygen partial pressure, electrical impedance, temperature, and light scattering and absorption properties including deoxygenated hemoglobin, vascularization, and tissue density. Because of the accuracy benefits from the six simultaneous measurements, and real-time nature of the device, it is expected to exceed the accuracy levels achieved by the core needle biopsy procedure and approach the high level of accuracy associated with surgical biopsies. Further, if cancer is found, the device can be configured to deliver various therapies such as cancer markers, laser heat, cryogenics, drugs, and radioactive seeds. Such devices are disclosed, for example, at www.bioluminate.com/description.html, which is incorporated herein by reference; and

(7) Personal UV-B recorders, which are instrument grade devices for measuring and recording UVB exposure and fit into a wrist-watch face. They may also be worn as a patch.

The shell (or coating) of the present invention may comprise any material which can be molded, including for example, film formers, low-melting hydrophobic materials, gelling polymers, thickeners, thermoplastic materials, non-crystallizable carbohydrates, plasticizers, adjuvants, and excipients.

In certain preferred embodiments of the invention, the shell is prepared by molding. In such embodiments, the shell is made from a flowable material. The flowable material may be any edible material that is flowable at a temperature between about 37° C. and 250° C., and that is solid, semi-solid, or can form a gel at a temperature between about −10° C. and about 35° C. When it is in the fluid or flowable state, the flowable material may comprise a dissolved or molten component, and optionally a solvent such as for example water or organic solvents, or combinations thereof. The solvent may be partially or substantially removed by drying. Suitable flowable materials include those comprising film formers; thickeners such as gelling polymers or hydrocolloids; thermoplastic materials; low melting hydrophobic materials such as fats and waxes; non-crystallizable carbohydrates; and the like. In one embodiment, the shell preferably comprises at least about 50%, preferably at least about 80%, most preferably at least about 90% of a material selected from film formers, gelling polymers, thermoplastic materials, low-melting hydrophobic materials, non-crystallizable sugars or sugar alcohols, and mixtures thereof. In another embodiment, the shell comprises at least about 50%, preferably at least about 80%, most preferably at least about 90% of a material selected from film formers, gelling polymers, low-melting hydrophobic materials, and mixtures thereof.

Any film former known in the art is suitable for use in the flowable shell material of the present invention. Examples of suitable film formers include, but are not limited to, polyvinylalcohol (PVA), hydroxypropyl starch, hydroxyethyl starch, pullulan, methylethyl starch, carboxymethyl starch, methylcellulose, hydroxypropylcellulose (HPC), hydroxyethylmethylcellulose (HEMC), hydroxypropylmethylcellulose (HPMC), hydroxybutylmethylcellulose (HBMC), hydroxyethylethylcellulose (HEEC), hydroxyethylhydroxypropylmethyl cellulose (HEMPMC), methacrylic acid and methacrylate ester copolymers, polyvinyl alcohol and polyethylene glycol copolymers, polyethylene oxide and polyvinylpyrrolidone copolymers, gelatin, proteins such as whey protein, coaggulatable proteins such as albumin, casein, and casein isolates, soy protein and soy protein isolates, pre-gelatinized starches, film-forming modified starches, and polymers, derivatives and mixtures thereof.

One suitable hydroxypropylmethylcellulose compound is “HPMC 2910”, which is a cellulose ether having a degree of substitution of about 1.9 and a hydroxypropyl molar substitution of 0.23, and containing, based upon the total weight of the compound, from about 29% to about 30% methoxyl groups and from about 7% to about 12% hydroxylpropyl groups. HPMC 2910 is commercially available from the Dow Chemical Company under the tradename METHOCEL E. METHOCEL E5, which is one grade of HPMC-2910 suitable for use in the present invention, has a viscosity of about 4 to 6 cps (4 to 6 millipascal-seconds) at 20° C. in a 2% aqueous solution as determined by a Ubbelohde viscometer. Similarly, METHOCEL E6, which is another grade of HPMC-2910 suitable for use in the present invention, has a viscosity of about 5 to 7 cps (5 to 7 millipascal-seconds) at 20° C. in a 2% aqueous solution as determined by a Ubbelohde viscometer. METHOCEL E15, which is another grade of HPMC-2910 suitable for use in the present invention, has a viscosity of about 15000 cps (15 millipascal-seconds) at 20° C. in a 2% aqueous solution as determined by a Ubbelohde viscometer. As used herein, “degree of substitution” shall mean the average number of substituent groups attached to a anhydroglucose ring, and “hydroxypropyl molar substitution” shall mean the number of moles of hydroxypropyl per mole anhydroglucose.

One suitable polyvinyl alcohol and polyethylene glycol copolymer is commercially available from BASF Corporation under the tradename KOLLICOAT IR.

As used herein, “modified starches” include starches that have been modified by crosslinking, chemically modified for improved stability or optimized performance, or physically modified for improved solubility properties or optimized performance. Examples of chemically-modified starches are well known in the art and typically include those starches that have been chemically treated to cause replacement of some of its hydroxyl groups with either ester or ether groups. Crosslinking, as used herein, may occur in modified starches when two hydroxyl groups on neighboring starch molecules are chemically linked. As used herein, “pre-gelatinized starches” or “instantized starches” refers to modified starches that have been pre-wetted, then dried to enhance their cold-water solubility. Suitable modified starches are commercially available from several suppliers such as, for example, A. E. Staley Manufacturing Company, and National Starch & Chemical Company. One suitable film forming modified starch includes the pre-gelatinized waxy maize derivative starches that are commercially available from National Starch & Chemical Company under the tradenames PURITY GUM and FILMSET, and derivatives, copolymers, and mixtures thereof. Such waxy maize starches typically contain, based upon the total weight of the starch, from about 0 percent to about 18 percent of amylose and from about 100% to about 88% of amylopectin.

Another suitable film forming modified starch includes the hydroxypropylated starches, in which some of the hydroxyl groups of the starch have been etherified with hydroxypropyl groups, usually via treatment with propylene oxide. One example of a suitable hydroxypropyl starch that possesses film-forming properties is available from Grain Processing Company under the tradename, PURE-COTE B790.

Suitable tapioca dextrins for use as film formers include those available from National Starch & Chemical Company under the tradenames CRYSTAL GUM or K-4484, and derivatives thereof such as modified food starch derived from tapioca, which is available from National Starch and Chemical under the tradename PURITY GUM 40, and copolymers and mixtures thereof.

Any thickener known in the art is suitable for use in the flowable material of the present invention. Examples of such thickeners include but are not limited to hydrocolloids (also referred to herein as gelling polymers) such as alginates, agar, guar gum, locust bean, carrageenan, tara, gum arabic, tragacanth, pectin, xanthan, gellan, maltodextrin, galactomannan, pusstulan, laminarin, scleroglucan, gum arabic, inulin, pectin, whelan, rhamsan, zooglan, methylan, chitin, cyclodextrin, chitosan, clays, gelling starches such as acid hydrolyzed starches, and derivatives and mixtures thereof. Additional suitable thickening hydrocolloids include low-moisture polymer solutions such as mixtures of gelatin and other hydrocolloids at water contents up to about 30%, such as for example those used to make “gummi” confection forms. Additional suitable thickeners include crystallizable carbohydrates, and the like, and derivatives and combinations thereof. Suitable crystallizable carbohydrates include the monosaccharides and the oligosaccharides. Of the monosaccharides, the aldohexoses e.g., the D and L isomers of allose, altrose, glucose, mannose, gulose, idose, galactose, talose, and the ketohexoses e.g., the D and L isomers of fructose and sorbose along with their hydrogenated analogs: e.g., glucitol (sorbitol), and mannitol are preferred. Of the oligosaccharides, the 1,2-disaccharides sucrose and trehalose, the 1,4-disaccharides maltose, lactose, and cellobiose, and the 1,6-disaccharides gentiobiose and melibiose, as well as the trisaccharide raffinose are preferred along with the isomerized form of sucrose known as isomaltulose and its hydrogenated analog isomalt. Other hydrogenated forms of reducing disaccharides (such as maltose and lactose), for example, maltitol and lactitol are also preferred. Additionally, the hydrogenated forms of the aldopentoses: e.g., D and L ribose, arabinose, xylose, and lyxose and the hydrogenated forms of the aldotetroses: e.g., D and L erythrose and threose are preferred and are exemplified by xylitol and erythritol, respectively.

In one embodiment of the invention, the flowable material comprises gelatin as a gelling polymer. Gelatin is a natural, thermogelling polymer. It is a tasteless and colorless mixture of derived proteins of the albuminous class which is ordinarily soluble in warm water. Two types of gelatin—Type A and Type B—are commonly used. Type A gelatin is a derivative of acid-treated raw materials. Type B gelatin is a derivative of alkali-treated raw materials. The moisture content of gelatin, as well as its Bloom strength, composition and original gelatin processing conditions, determine its transition temperature between liquid and solid. Bloom is a standard measure of the strength of a gelatin gel, and is roughly correlated with molecular weight. Bloom is defined as the weight in grams required to move a half-inch diameter plastic plunger 4 mm into a 6.67% gelatin gel that has been held at 10° C. for 17 hours. In a preferred embodiment, the flowable material is an aqueous solution comprising 20% 275 Bloom pork skin gelatin, 20% 250 Bloom Bone Gelatin, and approximately 60% water.

Suitable xanthan gums include those available from C.P. Kelco Company under the tradenames KELTROL 1000, XANTROL 180, or K9B310.

Suitable thermoplastic materials can be molded and shaped when heated, and include both water soluble and water insoluble polymers that are generally linear, not crosslinked, nor strongly hydrogen bonded to adjacent polymer chains. Examples of suitable thermoplastic materials include: chemically modified cellulose derivatives such as hydroxypropyl cellulose (HPC), hydroxypropylmethyl cellulose (HPMC), methyl cellulose (MC), cellulose acetate (CA), ethyl cellulose (EC), cellulose acetate butyrate (CAB), cellulose propionate; vinyl polymers such as polyvinyl alcohol (PVA) and polyvinyl pyrrolidone (PVP); thermoplastic starch; natural and chemically modified proteins such as gelatin, soy protein isolates, whey protein, myofibrillar proteins, and the milk derived caseinate proteins; and derivatives and combinations thereof. Other suitable thermoplastic materials include sugar in the form on an amorphous glass such as that used to make hard candy forms.

Suitable clays include smectites such as bentonite, kaolin, and laponite; magnesium trisilicate, magnesium aluminum silicate, and the like, and derivatives and mixtures thereof.

“Acid-hydrolyzed starch,” as used herein, is one type of modified starch that results from treating a starch suspension with dilute acid at a temperature below the gelatinization point of the starch. During the acid hydrolysis, the granular form of the starch is maintained in the starch suspension, and the hydrolysis reaction is ended by neutralization, filtration and drying once the desired degree of hydrolysis is reached. As a result, the average molecular size of the starch polymers is reduced. Acid-hydrolyzed starches (also known as “thin boiling starches”) tend to have a much lower hot viscosity than the same native starch as well as a strong tendency to gel when cooled.

“Gelling starches,” as used herein, include those starches that, when combined with water and heated to a temperature sufficient to form a solution, thereafter form a gel upon cooling to a temperature below the gelation point of the starch. Examples of gelling starches include, but are not limited to, acid hydrolyzed starches such as that available from Grain Processing Corporation under the tradename PURE-SET B950; hydroxypropyl distarch phosphate such as that available from Grain Processing Corporation under the tradename, PURE-GEL B990, and mixtures thereof.

Suitable low-melting hydrophobic materials may comprise sucrose-fatty acid esters; fats such as cocoa butter, hydrogenated vegetable oil such as palm kernel oil, cottonseed oil, sunflower oil, and soybean oil; free fatty acids and their salts; mono- di- and triglycerides, phospholipids, waxes such as carnuba wax, spermaceti wax, beeswax, candelilla wax, shellac wax, microcrystalline wax, and paraffin wax; fat-containing mixtures such as chocolate.

Suitable non-crystallizable carbohydrates include non-crystallizable sugars such as polydextrose, and starch hydrolysates, e.g. glucose syrup, corn syrup, and high fructose corn syrup; and non-crystallizable sugar-alcohols such as maltitol syrup.

Other suitable flowable materials include sugar in a supersaturated solution such as that used to make fondant forms.

The flowable material for making the core or the shell by molding may optionally comprise adjuvants or excipients, which may comprise up to about 20% by weight of the flowable material. Examples of suitable adjuvants or excipients include plasticizers, detackifiers, humectants, surfactants, anti-foaming agents, colorants, flavorants, sweeteners, opacifiers, and the like. In one preferred embodiment, the flowable material comprises less than 5% humectants, or alternately is substantially free of humectants, such as glycerin, sorbitol, maltitol, xylitol, or propylene glycol. Humectants have traditionally been included in pre-formed films employed in enrobing processes, such as that disclosed in U.S. Pat. Nos. 5,146,730 and 5,459,983, to ensure adequate flexibility or plasticity and bondability of the film during processing. Humectants function by binding water and retaining it in the film. Pre-formed films used in enrobing processes can typically comprise up to 45% water. Disadvantageously, the presence of humectant prolongs the drying process, and can adversely affect the stability of the finished dosage form.

Any plasticizer known in the pharmaceutical art is suitable for use in the present invention, and may include, but not be limited to polyethylene glycol; glycerin; sorbitol; triethyl citrate; tribuyl citrate; dibutyl sebecate; vegetable oils such as castor oil; surfactants such as polysorbates, sodium lauryl sulfates, and dioctyl-sodium sulfosuccinates; propylene glycol; mono acetate of glycerol; diacetate of glycerol; triacetate of glycerol; natural gums and mixtures thereof. In solutions containing a cellulose ether film former, an optional plasticizer may be present in an amount, based upon the total weight of the solution, from about 0% to about 40%. In certain embodiments, the shell is substantially free of plasticizers, i.e. contains less than about 1%, say less than about 0.01% of plasticizers.

In a preferred embodiment of the invention, the finished shell of the dosage form comprises at least about 80%, preferably at least about 90% of a material selected from film formers, gelling polymers (hydrocolloids), thermoplastic materials, low-melting hydrophobic materials, non-crystallizable sugars, and mixtures thereof. The shell of the present invention may be formed by injection molding, advantageously minimizing or eliminating the need for direct-compression filler-binders such as microcrystalline cellulose, spray-dried lactose, mineral salts such as calcium phosphate, crystalline sugars such as sucrose, dextrates and the like. These materials would disadvantageously detract from the clarity and stability of the shell. Preferably the shell of the present invention comprises less than about 10%, e.g. less than about 1%, or less than about 0.1% of direct-compression filler-binders. The shells of the present invention are thus an improvement over compression-coated shells, which typically comprise at least about 30% of a direct-compression filler-binder as disclosed, for example, in WO 00/18447.

In another preferred embodiment of the invention, the shell comprises any of the compositions described in pending U.S. patent application Serial No. ______ [attorney docket No. MCP320] which is incorporated herein by reference.

In another preferred embodiment of the invention, the shell comprises any of the compositions described in pending U.S. patent application Ser. No. ______ [attorney docket No. MCP321] which is incorporated herein by reference.

In another preferred embodiment of the invention, the flowable material comprises a film former such as a cellulose ether, e.g. hydroxypropylmethylcellulose or a modified starch, e.g. waxy maize starch; optionally an extender, such as polycarbohydrates, e.g. maltodextrin; optionally a thickener, such as a hydrocolloid, e.g. xanthan gum or carrageenan, or a sugar, e.g. sucrose; optionally a plasticizer, e.g. polyethylene glycol, propylene glycol, vegetable oils such as castor oil, glycerin, and mixtures thereof.

In yet another preferred embodiment, the shell contains, based upon the total dry solids weight of the shell composition, from about 25 percent to about 80 percent, e.g. from about 50 to about 75 percent, of a film former such as a chemically modified starch, e.g. hydroxypropyl starch; from about 0.10 percent to about 33 percent, e.g. from about 0.15 percent to about 1 percent, or from about 10 percent to about 25 percent of a thickening agent; and from about 11 percent to about 60 percent, e.g. from about 20 percent to about 40 percent of a plasticizer.

In one embodiment wherein the film former is a chemically modified starch, the thickener may be selected from the group consisting of kappa or iota carrageenan, maltodextrin, gellan gum, agar, gelling starch and derivatives and mixtures thereof.

In one embodiment wherein the film former is a chemically modified starch, the plasticizer may be selected from the group consisting of glycerin, propylene glycol, polyethylene glycol, sugar alcohols and derivatives and mixtures thereof.

In certain other preferred embodiments of the invention, the shell is substantially free of gelatin, i.e. contains less than about 1%, or less than about 0.01% of gelatin.

In certain other embodiments, the shell is substantially free of bovine derived materials, i.e. contains less than about 1%, or less than about 0.01% of bovine derived materials.

In a preferred embodiment of the invention, the shell is applied to the core in the form of a flowable material using the thermal cycle method and apparatus described in copending U.S. patent application Ser. No. 09/966,497, pages 27-51, the disclosure of which is incorporated herein by reference. In this embodiment, the shell is applied using a thermal cycle molding module having the general configuration shown in FIG. 3 therein. The thermal cycle molding module 200 comprises a rotor 202 around which a plurality of mold units 204 are disposed. The thermal cycle molding module includes a reservoir 206 (see FIG. 4 therein) for holding shell flowable material. In addition, the thermal cycle molding module is provided with a temperature control system for rapidly heating and cooling the mold units. FIGS. 55 and 56 depict the temperature control system 600.

The thermal cycle molding module is preferably of the type shown in FIG. 28A of copending U.S. application Ser. No. 09/966,497, comprising a series of mold units 204. The mold units 204 in turn comprise upper mold assemblies 214, rotatable center mold assemblies 212 and lower mold assemblies 210 as shown in FIG. 28C. Cores are continuously transferred to the mold assemblies, which then close over the cores. The shell flowable material, which is heated to a flowable state in reservoir 206, is injected into the mold cavities created by the closed mold assemblies. The temperature of the shell flowable material is then decreased, hardening it. The mold assemblies open and eject the coated cores. In one particular embodiment, coating is performed in two steps, each half of the cores being coated separately as shown in the flow diagram of FIG. 28B of copending U.S. application Ser. No. 09/966,497 via rotation of the center mold assembly.

In another embodiment, the shell may be prepared using thermal setting molding as described in copending U.S. patent application Ser. No. 09/966,450, pages 57-63.

The shell of the present invention may have a variable thickness, i.e. the thickness may be different at various points on the shell. In general, the shell thickness at any given point may preferably be from about 20 microns to about 30,000 microns, for example from about 50 to about 500 microns, say from about 50 microns to about 125 microns; or from about 100 microns to about 1000 microns, say from about 100 microns to about 400 microns; or from about 500 microns to about 30,000 microns; say from about 500 microns to about 2,000 microns.

In one embodiment, the shell of the present invention advantageously preferably has a high surface gloss. The surface gloss of the shell and/or finished dosage form is preferably at least about 150 gloss units, e.g. at least about 175 gloss units, or at least about 210 gloss units when measured by the method set forth below:

Dosage forms may be tested for surface gloss using an instrument available from TriCor Systems Inc. (Elgin, Ill.) under the tradename TRI-COR MODEL 805A/806H SURFACE ANALYSIS SYSTEM and generally in accordance with the procedure described in “TriCor Systems WGLOSS 3.4 Model 805A/806H Surface Analysis System Reference Manual” (1996), which is incorporated by reference herein, except as modified below.

This instrument uses a CCD camera detector, a flat diffuse light source, compares tablet samples to a reference standard, and determines average gloss values at a 60 degree incident angle. During its operation, the instrument generates a gray-scale image, wherein the occurrence of brighter pixels indicates the presence of more gloss at that given location.

The instrument also incorporates software that uses a grouping method to quantify gloss: i.e., pixels with similar brightness which are grouped together for averaging purposes.

The “percent full scale” or “percent ideal” setting (also referred to as the “percent sample group” setting), is specified by the user to designate the portion of the brightest pixels above the threshold that will be considered as one group and averaged within that group. “Threshold,” as used herein, is defined as the maximum gloss value that will not be included in the average gloss value calculation. Thus, the background, or the non-glossy areas of a sample are excluded from the average gloss value calculations. The method disclosed in K. Fegley and C. Vesey, “The Effect of Tablet Shape on the Perception of High Gloss Film Coating Systems,” which is available at www.colorcon.com as of Mar. 18, 2002 and incorporated by reference herein, is used to minimize the effects resulting from different tablet shapes, and to report a metric that was comparable across the industry. (The 50% sample group setting is selected as the setting which best approximates analogous data from tablet surface roughness measurements.)

After initially calibrating the instrument using a calibration reference plate (190-228; 294 degree standard; no mask, rotation 0, depth 0), a standard surface gloss measurement is created. For example, a standard surface gloss was obtained using gel-coated caplets available from McNEIL-PPC, Inc. under the tradename, EXTRA STRENGTH TYLENOL GELCAPS. The average gloss value for a sample of 112 of such gel-coated caplets was then determined, while employing the 25 mm full view mask (190-280), and configuring the instrument to the following settings:

Rotation: 0

Depth: 0.25 inches

Gloss Threshold: 95

% Full Scale: 50%

Index of Refraction: 1.57

The average surface gloss value for the reference standard was determined to be 269.

Dosage forms with high surface gloss are preferred by consumers due to their aesthetic elegance and perceived swallowability. The surface gloss of the shell depends upon a number of factors, including the shell composition, the method of forming the shell, and, if a mold is used, the surface finish on the mold.

One or more active ingredients may be contained in the dosage form of the invention in the core, the shell, the insert, or any combination thereof. In one embodiment of the invention, only the core comprises one or more active ingredients. In another embodiment of this invention, only the shell comprises one or more active ingredients. In yet another embodiment of this invention, only the insert comprises one or more active ingredients. In yet another embodiment of this invention, both the core and shell comprise one or more active ingredients. In yet another embodiment of this invention, one or more of the core, the shell, or the insert comprises one or more of the active ingredients.

Molded cores, shells or inserts as used in this invention preferably are substantially free of pores having a diameter of 0.5-5.0 microns. As used herein, “substantially free” means that the first molded material has a pore volume of less than about 0.02 cc/g, preferably less than about 0.01 cc/g, more preferably less than about 0.005 cc/g, in the pore diameter range of 0.5 to 5.0 microns. Typical compressed materials have pore volumes of more than about 0.02 cc/g in this pore diameter range. Pore volume, pore diameter and density may be determined using a Quantachrome Instruments PoreMaster 60 mercury intrusion porosimeter and associated computer software program known as “Porowin.” The procedure is documented in the Quantachrome Instruments PoreMaster Operation Manual. The PoreMaster determines both pore volume and pore diameter of a solid or powder by forced intrusion of a non-wetting liquid (mercury), which involves evacuation of the sample in a sample cell (penetrometer), filling the cell with mercury to surround the sample with mercury, applying pressure to the sample cell by: (i) compressed air (up to 50 psi maximum); and (ii) a hydraulic (oil) pressure generator (up to 60000 psi maximum). Intruded volume is measured by a change in the capacitance as mercury moves from outside the sample into its pores under applied pressure. The corresponding pore size diameter (d) at which the intrusion takes place is calculated directly from the so-called “Washburn Equation”: d=−(4γ(cos θ)/P where γ is the surface tension of liquid mercury, θ is the contact angle between mercury and the sample surface and P is the applied pressure.

Equipment used for pore volume measurements:

1. Quantachrome Instruments PoreMaster 60.

2. Analytical Balance capable of weighing to 0.0001 g.

3. Desiccator.

Reagents used for measurements:

1. High purity nitrogen.

2. Triply distilled mercury.

3. High pressure fluid (Dila AX, available from Shell Chemical Co.).

4. Liquid nitrogen (for Hg vapor cold trap).

5. Isopropanol or methanol for cleaning sample cells.

6. Liquid detergent for cell cleaning.

Procedure:

The samples remain in sealed packages or as received in the dessicator until analysis. The vacuum pump is switched on, the mercury vapor cold trap is filled with liquid nitrogen, the compressed gas supply is regulated at 55 psi., and the instrument is turned on and allowed a warm up time of at least 30 minutes. The empty penetrometer cell is assembled as described in the instrument manual and its weight is recorded. The cell is installed in the low pressure station and “evacuation and fill only” is selected from the analysis menu, and the following settings are employed:

Fine Evacuation time: 1 min.

Fine Evacuation rate: 10

Coarse Evacuation time: 5 min.

The cell (filled with mercury) is then removed and weighed. The cell is then emptied into the mercury reservoir, and two tablets from each sample are placed in the cell and the cell is reassembled. The weight of the cell and sample are then recorded. The cell is then installed in the low-pressure station, the low-pressure option is selected from the menu, and the following parameters are set:

Mode: Low pressure

Fine evacuation rate: 10

Fine evacuation until: 200 μHg

Coarse evacuation time: 10 min.

Fill pressure: Contact +0.1

Maximum pressure: 50

Direction: Intrusion And Extrusion

Repeat: 0

Mercury contact angle; 140

Mercury surface tension: 480

Data acquisition is then begun. The pressure vs. cumulative volume-intruded plot is displayed on the screen. After low-pressure analysis is complete, the cell is removed from the low-pressure station and reweighed. The space above the mercury is filled with hydraulic oil, and the cell is assembled and installed in the high-pressure cavity. The following settings are used:

Mode: Fixed rate

Motor speed: 5

Start pressure: 20

End pressure: 60,000

Direction: Intrusion and extrusion

Repeat: 0

Oil fill length: 5

Mercury contact angle: 140

Mercury surface tension: 480

Data acquisition is then begun and graphic plot pressure vs. intruded volume is displayed on the screen. After the high pressure run is complete, the low-and high-pressure data files of the same sample are merged.

In another embodiment of the invention, the core is coated with two or more shell portions, which may optionally be visually distinct, or compositionally or functionally different, from one another. As used herein, the term “compositionally different” means having features that are readily distinguishable by qualitative or quantitative chemical analysis, physical testing, or visual observation. For example, the first and second shell portions may contain different ingredients, or different levels of the same ingredients, or the first and second shell portions may have different physical or chemical properties, different functional properties, or be visually distinct. Examples of physical or chemical properties that may be different include hydrophylicity, hydrophobicity, hygroscopicity, elasticity, plasticity, tensile strength, crystallinity, and density. Examples of functional properties which may be different include rate and/or extent of dissolution of the material itself or of an active ingredient therefrom, rate of disintegration of the material, permeability to active ingredients, permeability to water or aqueous media, and the like. Examples of visual distinctions include size, shape, topography, or other geometric features, color, hue, opacity, and gloss. In one such embodiment, the first and second shell materials may be visually distinct from one another, for example the visually distinct portions may be of different colors, hues, glosses, reflective qualities, brightness, depth, shades, chroma, opacity, etc. For example, the shell may have a red portion and a yellow portion, or a flat finish portion and a glossy portion, or an opaque portion and a translucent portion. In another such embodiment, the first and second shell portions may comprise different shell materials. For example, the first and second shell portions may comprise different colorants, opacifiers, film-formers, etc. In another such embodiment, the first and second shell portions may have different functionality. For example, the first shell portion may function as a diffusional membrane which contains pores through which fluids can enter the dosage form, and dissolved drug can be released from an underlying core portion. In preferred embodiments in which a shell portion functions as a diffusional membrane, the release of the drug from the dosage form may be described as controlled, prolonged, sustained, extended. In these embodiments, the contribution to drug dissolution from the subject shell portion may follow zero-order, first-order, or square-root of time kinetics. The second shell portion, for example, may fiction as an eroding matrix from which drug dispersed in the second shell portion is liberated by the dissolution of successive layers of the shell portion surface.

In another embodiment of the invention, the shell may comprise two shell portions, and each shell portion has a different topography. For example, in a particularly preferred embodiment a first shell portion has perforations or holes therein, and a second shell portion has a smooth exterior surface with no perforations or holes therein. In another embodiment, a first shell portion has indentations therein, and a second shell portion has a smooth exterior surface with no indentations therein.

In another particularly preferred embodiment one or both shell portions have “Braille bumps” on the exterior surface, to enable blind persons to identify the contents of the dosage form.

The dosage form of the invention may also be constructed to impart regular or irregular, continuous or discontinuous, coatings or shells (i.e. of various portions and patterns) to the core. For example, dimple patterned shells, similar to the surface of a golf ball, can be provided. Alternatively, a circumferential portion of a core can be coated with one flowable material and the remaining portions of the core with another flowable material. Still another example of an irregular shell is a discontinuous coating comprising holes of uncoated portions around the core.

Embossments or debossments (in the form of letters, symbols, and the like) can also be provided onto the core. In either case, coating material may be selectively applied to either the unembossed or undebossed surface of the core, or to the surface of the embossments of debossments. In one embodiment a first coating (or shell portion) may be applied to cover the unembossed or undebosssed surface of the core but not the surface of the embossments or debossments, and a second coating (or shell portion) may be applied to cover the surface of the embossments or debossments. Optionally, the first and second coating materials may be visually distinct from one another.

Alternatively, only a portion of the core may be coated while the remainder is uncoated.

In one preferred embodiment, the invention provides a dosage form comprising a core having an injection molded shell surrounding at least a portion of the core.

In another preferred embodiment, the invention provides a dosage form comprising a core having a thermal cycle molded shell material disposed on at least a portion of the core.

In another preferred embodiment, the invention provides a dosage form comprising a core having a thermal setting molded shell material disposed on at least a portion of the core.

In another preferred embodiment, the invention provides a dosage form comprising an active ingredient in which the dosage form is prepared by molding a flowable material and the dosage form has no more than one plane of symmetry.

In another embodiment of the invention, the dosage form comprises a smooth shell applied to a core that is irregular in topography. Typical compressed tablets that are regular in topography, i.e. have a smooth surface, may have indentations or protrusions at the microscopic level on the order of less than about 20 microns in width, depth, or height. As used herein, the term “irregular in topography” shall apply to cores having indentations or protrusions greater than about 20 microns, preferably greater than about 50 microns, more preferably greater than about 100 microns, most preferably greater than about 1000 microns in width, depth, or height. In this embodiment, the outer surface of the shell surrounding the core of the dosage form can be made to be highly regular and smooth, even if the core itself is not. The dosage forms of this embodiment typically comprise a core with a surface having indentations or protrusions, surrounded by a shell having a thickness, wherein the ratio of the width of one or more indentations or protrusions to the thickness of the shell at one or more locations is at least about 1:1, e.g. at least about 2:1, or at least about 3:1. Once coated, the relative standard deviations in thickness and diameter of the dosage form is typically not greater than about 2%, preferably not more than about 1%, most preferably not more than about 0.35%. Typical dosage form thicknesses (shown in FIG. 2 as t) are on the order of about 4 to 10 mm, while typical dosage form diameters (d in FIG. 2) range from about 5 to about 15 mm.

In another embodiment of the invention, the dosage form comprises a substantially smooth core, at least a portion of which is surrounded by a shell that is irregular in topography. For example at least a portion of the shell has indentations or protrusions greater than about 20 microns, preferably greater than about 50 microns, more preferably greater than about 100 microns, most preferably greater than about 1000 microns in width, depth, or height.

In another embodiment of the invention, the core has an outer surface, the shell has an inner surface and an outer surface, and the shell resides substantially conformally upon the core outer surface, such that the peaks and valleys of the inner surface of the shell correspond substantially inversely to the major peaks and valleys of the outer surface of the core, and the outer surface of the shell does not substantially conform to the major peaks and valleys of the outer surface of the core.

In another embodiment of the invention, the dosage form comprises a core having an outer surface with a first topography; and a shell having an outer surface with a second topography, wherein at least one of the first or second topographies includes indentations or protrusions greater than about 20 microns in width, depth or height, and the first topography is different than the second topography.

In another embodiment of the invention, the dosage form comprises a core having an outer surface having indentations or protrusions greater than about 20 microns in width, depth, or height; and a shell having an inner surface and an outer surface, and the shell resides substantially conformally upon the core outer surface, such that the inner surface of the shell has protrusions and indentations which correspond substantially inversely to the major protrusions and indentations of the outer surface of the core, and the outer surface of the shell does not substantially conform to the major protrusions and indentations of the outer surface of the core.

In another embodiment of the invention, the dosage form comprises a core having an outer surface having indentations or protrusions; a shell which surrounds the core, wherein the shell has an inner surface, an outer surface and a thickness, the ratio of the width of one or more indentations or protrusions in the core surface to the thickness of the shell at one or more locations is at least about 1:1, the shell resides substantially conformally upon the core outer surface such that the inner surface of the shell has protrusions and indentations which correspond substantially inversely to the major indentations and protrusions of the outer surface of the core, and the outer surface of the shell does not substantially conform to the major protrusions and indentations of the outer surface of the core.

As used herein, the term “substantially conformally” shall mean that the microscopic inner surface of the shell has peaks and valleys which correspond substantially inversely to the peaks and valleys of the microscopic outer surface of the core.

In one embodiment of this invention, the dosage form of the invention advantageously avoids visible defects in its outer shell surface. Known injection molding processes utilize sprues and runners to feed moldable material into the mold cavity. This results in product defects such as injector marks, sprue defects, gate defects, and the like. In conventional molds, sprues and runners must be broken off after solidification, leaving a defect at the edge of the part, and generating scrap. In conventional hot runner molds, sprues are eliminated, however a defect is produced at the injection point since the hot runner nozzle must momentarily contact the chilled mold cavity during injection. As the tip of the nozzle retracts it pulls a “tail” with it, which must be broken off. This defect is particularly objectionable with stringy or sticky materials. Unwanted defects of this nature would be particularly disadvantageous for swallowable dosage forms, not only from a cosmetic standpoint but functionally as well. The sharp and jagged edges would irritate or scratch the mouth, tongue and throat. The dosage form of this invention avoids these problems.

In another embodiment of the invention, the core is shaped to permit modified release of active material within the core upon breach of the shell, such that the active ingredient within the core is released in such a manner as to yield a modified release profile. As used herein, modified release shall include sustained release, extended release, prolonged release, delayed release, pulsatile release, or any release profile which is intentionally altered from the release profile obtained for immediate release tablets of the particular active ingredient employed. For example, as disclosed in U.S. Pat. No. 4,663,147, it is known that sustained or controlled release of an active ingredient into the body may be achieved by employing a diffusible solid containing an active ingredient and coated with a fluid impermeable polymer on the outer surface of the solid, in which the solid has a cavity therein which is not coated with the fluid impermeable polymer. When the solid is ingested, fluid such as water in the gastro-intestinal tract enters the cavity of the solid and causes the release of the active ingredient at a substantially controlled or constant rate.

In a preferred embodiment of this invention, at least a portion of the shell comprises one or more openings. These openings permit the passage of liquid through the shell and contacting of the liquid (e.g. water in the gastrointestinal tract) with the core. The openings may be provided for in the shell, for example, using a mold having an inner surface having projections or protrusions.

FIG. 3 depicts a dosage form 30 in accordance with this invention, which comprises a shell 38 having a shape which surrounds the outside surface of a core 32 having an aperture or cavity 33 therein. As shown, core 32 and shell 38 have substantially different shapes. In one embodiment, shell 38 is made of a water soluble material, core 32 contains an active ingredient, and all surfaces of core 32 except interior cavity surface 36 may be coated with a fluid impermeable polymer. Upon ingestion of dosage form 30, the shell 38 is breached by water, stomach acid, intestinal fluid or the like, and such fluid reaches opening 33 and contacts inner surface 36 of core 32 as well as the fluid impermeable surfaces of core 32. Thus, active ingredient is released from the inner surface 36 of core 32 but not from the other core surfaces, and the ratio of surface area of diffusible solid in core 32 exposed to the fluid medium to the length of the path through which the exposed solid must diffuse to exit the core remains substantially constant, thereby providing a constant or controlled release of active ingredient. In one preferred embodiment, the shell surrounds the entire core including the aperture (as shown in FIG. 3).

In another embodiment of this invention, as depicted in FIGS. 4A and 4B, the core 440 has a first topography on its outer surface 441 and the shell 442 has a second topography on its outer surface 443 which is different from the first topography. More particularly, FIG. 4B depicts core outer surface 441 having protrusions 447 and indentations 448, thereby providing core outer surface 441 with a first topography. As is also shown in FIGS. 4A and 4B, the shell outer surface 443 comprises a plurality of protrusions 444 and indentations 445 as well as slits or cuts 446, thereby providing shell outer surface 443 with a second topography which is different than the first topography of core outer surface 441.

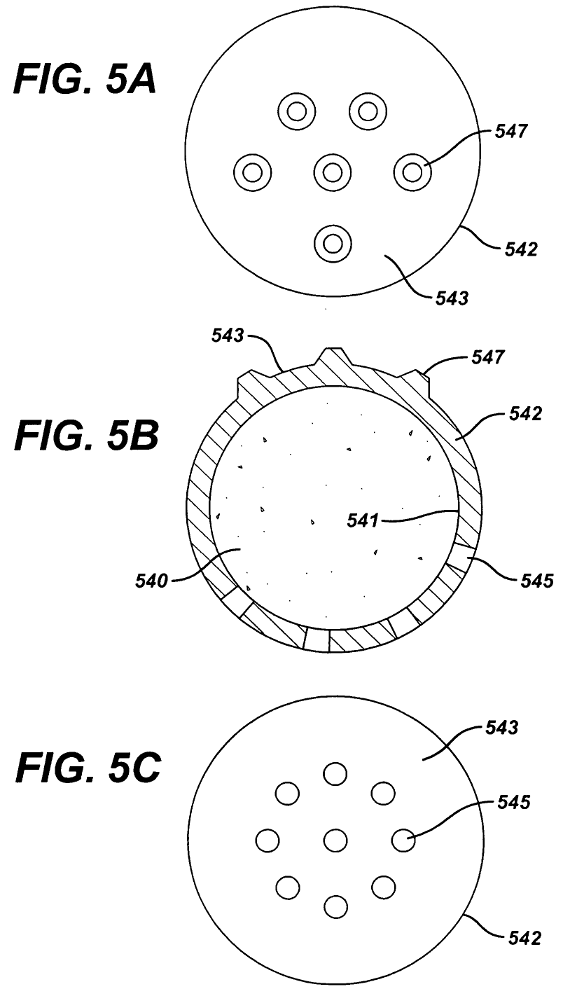

In another embodiment of this invention, as depicted in FIGS. 5A, 5B and 5C, the core 540 has a first topography on its outer surface 541 and the shell 542 has a different topography on its outer surface 543. More particularly, FIG. 5B depicts core outer surface 541 which is substantially smooth, thereby providing core outer surface 541 with a first topography. FIGS. 5A-5C depict the shell outer surface 543 having protrusions 547 and indentations 545, thereby providing shell outer surface 543 with a second topography which is different than the first topography of core outer surface 541.

In another embodiment of this invention, as depicted in FIGS. 6A, 6B and 6C, the core 640 has a visual image 645 comprising protrusions originating from the outer surface 641 of core 640, thereby giving outer surface 641 a first topography. The shell 642 has a smooth outer surface 643, and thus a different topography than core outer surface 641. More particularly, FIG. 6B depicts core outer surface 641 which has protrusions 650, 651 and 652 as well as protrusions 653, 654, 655, and 656.

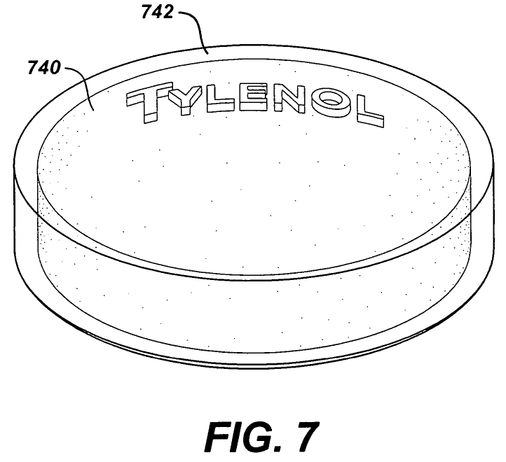

In another embodiment of this invention, as depicted in FIG. 7, the core 740 has information which is visually observable (i.e. the word “TYLENOL” in FIG. 7) embossed upon the core 740. Surrounding the core 740 is a shell 742. Thus, the core outer surface has a first topography due to the presence of the embossed information, and the shell outer surface has a second topography which is different than the first topography of the core outer surface.

In another embodiment of the invention, an insert is employed, and the insert is not completely contained within the core. For example, the insert may be larger than the core in at least one dimension or along at least one axis of the core, and thus the insert is partially contained within the core and partially contained within the shell. This is depicted in FIG. 8, which shows a dosage form 802 comprising a core 804 having a first shape and a shell 806 having a second shape which is substantially different than the first shape of core 804. Core 804 additionally comprises an insert 808 which is only partially contained within core 804, as shown in FIG. 8.

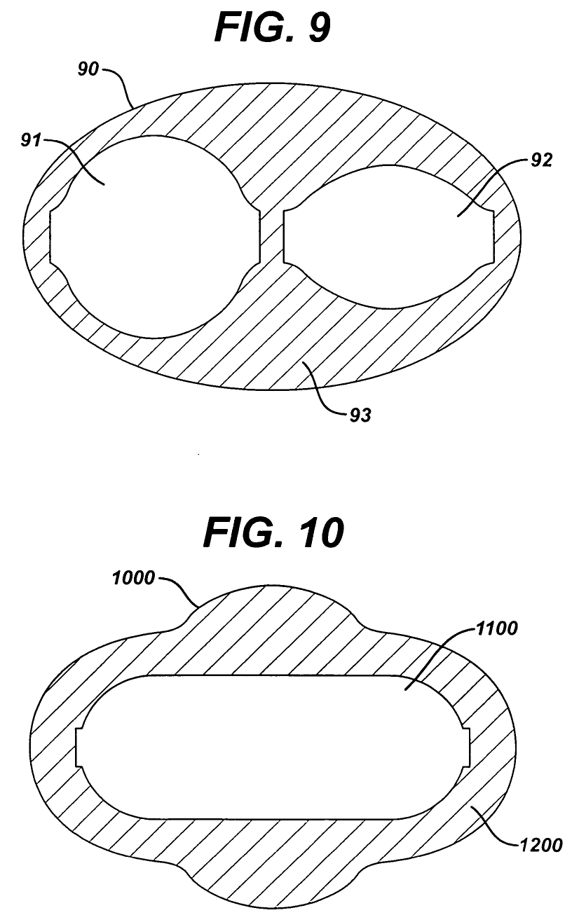

In another embodiment of the invention, the dosage form comprises more than one core, for example two cores surrounded by a single shell. This is depicted in FIG. 9, which shows a dosage form 90 comprising a first core 91 having a first shape; a second core 92 having a second shape; and a shell 93 having a third shape which is substantially different than either the first shape of the first core 91, or the second shape of the second core 92, or the combined shape of the two cores 91 and 92.

FIG. 10 depicts a cross-sectional view of another embodiment of this invention in which a dosage form 1000 contains a core 1100 having a first shape and a shell 1200 having a second shape which surrounds the core 1100, with the first shape and second shape being substantially different.

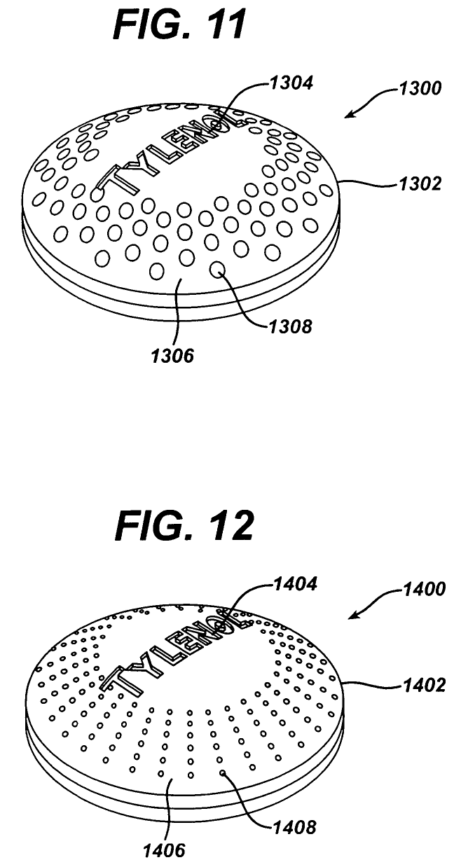

FIG. 11 depicts a side view of another embodiment of this invention in which the dosage form 1300 has a shell 1302 having a debossed region 1304 displaying a logo and another region 1306 comprising a plurality of large indentations 1308.

FIG. 12 depicts a side view of another embodiment of this invention in which the dosage form 1400 has a shell 1402 having a debossed region 1404 displaying a logo and another region 1406 comprising a plurality of smaller indentations 1408 which are in a different pattern than the larger indentations 1308 in FIG. 11.

This invention will be further illustrated by the following examples, which are not meant to limit the invention in any way.

### EXAMPLE 1

Dosage forms of the invention are made in a continuous process using an apparatus comprising two thermal cycle molding modules linked in series via a transfer device as described at pages 14-16 of copending U.S. application Ser. No. 09/966,939, the disclosure of which is incorporated herein by reference. The dosage forms have the structure shown in FIG. 1A and each comprise a core having the shape of an elongated cross coated with a shell having an ellipsoidal shape.

The core is made of a core flowable material comprising the following ingredients:

| | | | Weight | Mg/ |
|---|---|---|---|---|
| Tablet | Trade Name | Manufacturer | % | Tablet |
| Polyethylene | Carbowax ® | Union Carbide | 60.3 | 190 |
| Glycol 3350 | | Corporation, | | |
| | | Danbury, CT | | |
| Croscarmellose | Ac-Di-Sol ® | FMC Corporation, | 30.1 | 95 |
| Sodium | | Newark, DE | | |
| Pseudoephedrine | | BASF | 9.5 | 30 |
| Hydrochloride | | PharmaChemikalien | | |
| Crystal | | GmbH & Co., | | |
| | | Ludwigshafen/ | | |
| | | Rhein. | | |

The shell is made from a shell flowable material comprising the following ingredient:

| | | | | Mg/Dosage |
|---|---|---|---|---|
| Shell | Trade Name | Manufacturer | Weight % | Form |
| Polyethylene | Carbowax ® | Union Carbide | 100 | 700 |
| Glycol | | Corporation, | | |
| 3350 | | Danbury, CT | | |

The thermal cycle molding modules have the general configuration shown in FIG. 3 of copending U.S. application Ser. No. 09/966,939, which depicts a thermal cycle molding module 200 comprising a rotor 202 around which a plurality of mold units 204 are disposed. Each thermal cycle molding module includes its own reservoir 206 (see FIG. 4 of copending U.S. application Ser. No. 09/966,939) for holding the core flowable material and the shell flowable material, respectively. In addition, each thermal cycle molding module is provided with a temperature control system for rapidly heating and cooling the mold units. FIGS. 55 and 56 of copending U.S. application Ser. No. 09/966,939 depict the temperature control system 600.

The cores are made in a first thermal cycle molding module, which is linked via a transfer device to a second thermal cycle molding module. The first thermal cycle molding module has the specific configuration shown in FIG. 26A of copending U.S. application Ser. No. 09/966,939. The first thermal cycle molding module comprises center mold assemblies 212 and upper mold assemblies 214 as shown in FIG. 26C, which mate to form mold cavities having the shape of an elongated cross. As rotor 202 rotates, the opposing center and upper mold assemblies close. Core flowable material, which is heated to a flowable state in reservoir 206, is injected into the resulting mold cavities. The temperature of the core flowable material is then decreased, hardening the core flowable material into cores. The mold assemblies open and eject the cores, which are received by the transfer device.

The transfer device has the structure shown as 300 in FIG. 3 of copending U.S. application Ser. No. 09/966,939. It comprises a plurality of transfer units 304 attached in cantilever fashion to a belt 312 as shown in FIGS. 68 and 69 of copending U.S. application Ser. No. 09/966,939. The transfer device rotates and operates in sync with the thermal cycle molding modules to which it is coupled. Transfer units 304 comprise retainers 330 for holding the cores as they travel around the transfer device.

The transfer device transfers the cores to the second thermal cycle molding module, which applies the shell to the cores. The second thermal cycle molding module is of the type shown in FIG. 28A of copending U.S. application Ser. No. 09/966,939. The mold units 204 of the second thermal cycle molding module comprise upper mold assemblies 214, rotatable center mold assemblies 212 and lower mold assemblies 210 as shown in FIG. 28C. Cores are continuously transferred to the mold assemblies, which then close over the cores. Shell flowable material, which is heated to a flowable state in reservoir 206, is injected into the mold cavities created by the closed mold assemblies. The temperature of the shell flowable material is then decreased, hardening it. The mold assemblies open and eject the coated cores. Coating is performed in two steps, each half of the cores being coated separately as shown in the flow diagram of FIG. 28B of copending U.S. application Ser. No. 09/966,939 via rotation of the center mold assembly.

Although this invention has been illustrated by reference to specific embodiments, it will be apparent to those skilled in the art that various changes and modifications may be made which clearly fall within the scope of this invention.

The invention claimed is:

1. A dosage form comprising: an active ingredient; a core having an outer surface and a first shape; and a shell having outer and inner surfaces and a second shape which is substantially different than the first shape, wherein the shell comprises at least about 80% of a flowable material selected from the group consisting of film formers, gelling polymers, thermoplastic materials, low melting hydrophobic materials, non-crystallizable sugars, non-crystallizable sugar alcohols, and mixtures thereof, and the shell surrounds at least a portion of the core.

2. A dosage form comprising: an active ingredient; a core having an outer surface with a first topography; and a shell having an inner surface and an outer surface with a second topography which is different than the first topography, wherein at least one of the first or second topographies includes indentations or protrusions greater than about 20 microns in width, depth or height, and the shell surrounds at least a portion of the core.

3. A dosage form comprising: an active ingredient; a core having an outer surface having indentations or protrusions greater than about 20 microns in width, depth, or height; and a shell having an inner surface and an outer surface, wherein the shell resides substantially conformally upon at least a portion of the core outer surface, such that the inner surface of the shell has protrusions and indentations corresponding substantially inversely to the major protrusions and indentations of the outer surface of the core, and the outer surface of the shell does not substantially conform to the major protrusions and indentations of the outer surface of the core.

4. A dosage form comprising: an active ingredient; a core having an outer surface having indentations or protrusions; and a shell which surrounds at least a portion of the core, wherein the shell has an inner surface, an outer surface and a thickness, the ratio of the width of one or more indentations or protrusions in the core surface to the thickness of the shell at one or more locations is at least about 1:1, the shell resides substantially conformally upon the core outer surface such that the inner surface of the shell has protrusions and indentations correspond substantially inversely to the major indentations and protrusions of the outer surface of the core, and the outer surface of the shell does not substantially conform to the major protrusions and indentations of the outer surface of the core.

5. A dosage form comprising: an active ingredient; a first core having an outer surface with a first topography; a second core having an outer surface with a second topography; and a shell having an inner surface and outer surface with a third topography which is different than the first topography, wherein at least one of the first, second, or third topographies includes indentations or protrusions greater than about 20 microns in width, depth or height, and the shell surrounds at least a portion of the core.

6. A dosage form comprising: an active ingredient; a core comprising a first and second core portion having outer surfaces with a first and second topographies, respectively; a first shell portion having an outer surface with a third topography; and a second shell portion having an outer surface with a fourth topography, wherein at least one of the third or fourth shell surface topographies is different from the underlying core portion topography, at lease one of the first, second, third, or fourth topographies includes indentations or protrusions greater than about 20 microns in width, depth or height, and the shell surrounds at least a portion of the core.

7. A dosage form comprising: an active ingredient; a core having an outer surface and a first shape; and a shell having outer and inner surfaces and a second shape which is substantially different than the first shape, wherein the shell is substantially free of pores having a pore diameter of 0.5 to 5.0 microns, and the shell surrounds at least a portion of the core.

8. The dosage form of any of claims 1-6, wherein the shell is substantially free of pores having a pore diameter of 0.5-5.0 microns.

9. The dosage form of any of claims 1-7, wherein the core and shell each have a different number of planes of symmetry with respect to the same reference axis.

10. The dosage form of any of claims 1-7, wherein the distance from the core outer surface to the shell outer surface is different at two different points located on the core outer surface, and the difference in distance is greater than about 125 microns.

11. The dosage form of claims 10, wherein the difference in distance is in the range of about 125-30,000 microns.

12. The dosage form of any of claims 1-7, wherein the shell comprises less than 10% by weight of a direct-compression filler-binder.

13. The dosage form of any of claims 1-7, in which the outer surface of the core displays written information, and the shell outer surface is transparent, semi-transparent or translucent.

14. The dosage form of any of claims 1-7, in which the outer surface of the shell displays written information.

15. The dosage form of any of claims 1-7, in which the shell is transparent, semi-transparent or translucent.

16. The dosage form of any of claims 1-7, in which the core and shell have different colors.

17. The dosage form of any of claims 1-7, in which the core is visually observable.

18. The dosage form of any of claims 1-7, in which the core, the shell, or both the core and shell comprise an active ingredient.

19. The dosage form of any claims 1-7, in which only the core comprises an active ingredient.

20. The dosage form of claim 18 or claim 19, in which the active ingredient is capable of dissolution, and dissolution of the active ingredient meets USP specifications for immediate release tablets containing the active ingredient.

21. The dosage form of any of claims 1-7, in which the core comprises a compressed dosage form.

22. The dosage form of any of claims 1-7, in which the core comprises a microelectronic device.

23. The dosage form of any of claims 1-7, in which the core comprises an insert.

24. The dosage form of claim 23, in which the insert is larger than the core in at least one dimension.

25. The dosage form of claim 23, in which at least a portion of the insert protrudes from the core.

26. The dosage form of claim 23, in which the insert comprises an active ingredient.

27. The dosage form of claim 26, in which the active ingredient is capable of dissolution, and dissolution of the active ingredient contained in the insert meets USP specifications for immediate release tablets containing the active ingredient.

28. The dosage form of claim 23, in which the insert comprises a microelectronic device.

29. The dosage form of any of claims 1-7, in which the outer surface of the shell is textured.

30. The dosage form of any of claims 1-7, in which the outer surface of the shell contains a prearranged pattern.

31. The dosage form of any of claims 1-7, in which the shell comprises one or more openings therein.

32. The dosage form of any of claims 1-7, in which the outer surface of the shell is substantially smooth.

33. The dosage form of any of claims 1-7, in which the shape of the core permits controlled release of core upon breach of the shell.

34. The dosage form of any of claims 1-7, in which the outer surface of the shell has a shape selected from the group consisting of spheres, ovoids, ellipses, and flattened derivatives thereof.

35. The dosage form of any of claims 1-7, in which the dosage form comprises a single core.

36. The dosage form of any of claims 1-7, in which the core and shell each have a major plane of symmetry, and the major plane of symmetry of the core is orthogonal to the major plane of symmetry of the shell.

37. The dosage form of any of claims 1-7, in which the core has an aperture therein defining an interior surface.

38. The dosage form of any of claims 1-7, in which the core is in the shape of a torus.

39. The dosage form of any of claims 1-7, in which the shell comprises first and second shell portions having first and second topographies respectively, and the first and second topographies are different.

40. The dosage form of claim 39, in which each of the first and second shell portions have an outer surface, and at least one of the outer surfaces comprises Braille symbols.

41. The dosage form of any of claims 1-7, in which the outer surface of the core contains indentations, intagliations, letters, symbols or a pattern.

42. The dosage form of claim 41, in which the shell covers a portion of the core, but does not substantially cover the indentations, intagliations, letters, symbols or pattern.

43. The dosage form of claim 41, in which a first shell portion coating covers the indentations, intagliations, letters, symbols or pattern but does not substantially cover the remaining portion of the core.

44. The dosage form of claim 43, in which a second shell portion covers the portion of the core which is not covered by the first shell portion.

45. The dosage form of any of claims 1-7, in which the outer surface of the core contains raised protrusions in the form of letters, symbols or a pattern.

46. The dosage form of claim 43, in which the shell covers a portion of the core, but does not substantially cover the raised protrusions.

47. The dosage form of claim 45, in which a first shell portion covers the raised protrusions, but does not substantially cover the remaining portion of the core.

48. The dosage form of claim 47, in which a second shell portion covers the portion of the core which is not covered by the first shell portion.

49. The dosage form of any of claims 1-7, in which the core outer surface is debossed or embossed with visual information and the shell outer surface is transparent, semi-transparent or translucent.

50. The dosage form of any of claims 1-7, wherein the shell contains, based upon the total dry solids weight of the shell composition, from about 25 percent to about 80 percent of a film former; from about 0.10 percent to about 33 percent of a thickening agent; and from about 11 percent to about 60 percent of a plasticizer.

51. The dosage form of any of claims 1-7, in which the dosage form further comprises a second core.

52. The dosage form of any of claims 1-7, wherein the shell has a topography which includes indentations or protrusions greater than about 20 microns in width, depth, or height.

53. The dosage form of any of claims 1-7, in which the outer surface of the shell contains a prearranged pattern.

54. The dosage form of claim 53 in which the prearranged pattern comprises Braille symbols.

55. The dosage form of any of claims 1-7, in which at least a portion of the shell comprises one or more openings therein.

56. The dosage form of claim 55 in which the shell comprises a plurality of openings therein.

57. The dosage form of claim 56 in which the openings form a prearranged pattern.

58. The dosage form of any of claims 1-7, in which the shell comprises first and second portions having first and second topographies, respectively, and the first and second topographies are different.

59. The dosage form of any of claims 1-7, in which at least a portion of the shell is transparent, semi-transparent, or translucent.

60. The dosage form of any of claims 1-7, wherein the shell contains, based upon the total dry solids weight of the shell composition, from about 25 percent to about 80 percent of a film former; from about 0.10 percent to about 33 percent of a thickening agent; and from about 11 percent to about 60 percent of a plasticizer.
